(19) United States
(12) Patent Application Publication
(10) Pub. No.: US 2023/0131170 A1
(43) Pub. Date: Apr. 27, 2023

(54) TOPICAL COMPOSITIONS AND METHODS FOR PHOTODYNAMIC THERAPY

(71) Applicant: DUSA Pharmaceuticals, Inc., Billerica, MA (US)

(72) Inventors: Pradeep SANGHVI, North Brunswick, NJ (US); Scott LUNDAHL, Billerica, MA (US); Xiaopin JIN, Mississauga (CA); Brenden B HADJIKEZIAN, Vaughan (CA); Jerzy ZADYKOWICZ, Mississauga (CA); Haydar ABDALGHAFOR, Brampton (CA); Tasnuva HAQUE, Brampton (CA)

(21) Appl. No.: 17/968,931

(22) Filed: Oct. 19, 2022

Related U.S. Application Data
(60) Provisional application No. 63/257,175, filed on Oct. 19, 2021.

(51) Int. Cl. A61K 41/00; A61K 31/197; A61K 9/00; A61K 47/18; A61K 47/10; A61K 47/34

(52) U.S. Cl. CPC A61K 41/0061; A61K 9/0014; A61K 31/197; A61K 47/10; A61K 47/34; A61K 47/183

(57) ABSTRACT

Methods and topical compositions for the treatment of a dermatological disorder are provided. The topical composition includes 5-aminolevulinic acid, at least one penetration enhancer and at least one chelating agent. Further, methods and topical compositions for use in photodynamic therapy are disclosed, in which the topical composition is applied to the affected area of a patient for a period in the range of about 15 minutes to about 10 hours.

### FIELD OF THE INVENTION

The present invention relates to a topical composition for the treatment of a dermatological disorder, comprising 5-aminolevulinic acid, at least one penetration enhancer and at least one chelating agent. The present invention further relates to the topical composition for use in a photodynamic therapy. The present invention also relates to methods for the treatment of dermatological disorders in a photodynamic therapy.

### BACKGROUND OF THE INVENTION

Photodynamic therapy (PDT), photodynamic diagnosis (PD), or photochemotherapy is generally used to treat and/or diagnose several types of ailments in or near the skin or other tissues, such as those in a body cavity. For example, photodynamic therapy or photodynamic diagnosis may be used for treatment or diagnosis of actinic keratosis of the upper extremities (e.g., the dorsal surface of the hand or forearms), scalp or facial areas of a patient. In addition, such techniques may be used for treatment and diagnosis of other indications (e.g., acne, warts, psoriasis, photo-damaged skin, cancer) and other areas of the patient (e.g., the legs or portions of the arms other than the forearms).

During one form of photodynamic therapy, a patient is first administered a photoactivatable agent or a precursor of a photoactivatable agent that accumulates in the tissue to be treated. The area in which the photoactivatable agent is administered is then exposed to visible light, which causes chemical and/or biological changes in the agent. These changes allow the agent to then selectively locate, destroy, or alter the target tissue while, at the same time, causing at most only mild and reversible damage to other tissues in the treatment area. One example of a precursor of a photoactivatable agent is 5-aminolevulinic acid (“ALA”), a 5-aminoketone compound of Formula I

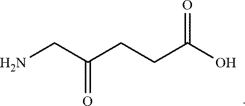

It is commonly used in photodynamic therapy of actinic keratosis. As they are used here, the terms ALA or 5-aminolevulinic acid refer to ALA itself, precursors thereof, esters thereof and pharmaceutically acceptable salts of the same. Photosensitization, following application of a topical composition (e.g., a topical solution or emulsion or nanoemulsion) containing ALA, occurs through the metabolic conversion of aminolevulinic acid to protoporphyrin IX (PpIX). PpIX is a photosensitizer which accumulates in the skin. ALA, which is formed from succinyl CoA and glycine in the first step of heme synthesis, is to a limited extent able to penetrate the skin and lead to a localised build-up of PpIX; since the action of ferrochelatase (the metallating enzyme) is the rate limiting step in heme synthesis, an excess of ALA leads to accumulation of PpIX, the photosensitizing agent. When exposed to light of appropriate wavelength and energy, the accumulated photoactive porphyrins produce a photodynamic reaction, resulting in a cytotoxic process dependent upon the simultaneous presence of oxygen. The absorption of light results in an excited state of porphyrin molecules, and subsequent spin transfer from photoreactive porphyrins to molecular oxygen generates singlet oxygen, which can further react to form superoxide and hydroxyl radicals. Since the skin covering actinic keratitis lesions, basilomas and squamous cell carcinomas are more readily penetrated by ALA than healthy skin, these changes allow the PpIX to then selectively locate, destroy, or alter the target tissue while, at the same time, causing at most only mild and reversible damage to other tissues in the treatment area.

For photodynamic therapy to be effective, it is desirable to have a power output that can be controlled for intensity and duration, among other factors. Illuminators are typically used to provide the proper uniformity of light for treatment purposes. These devices generally include a light source (e.g., a fluorescent tube or LED), coupling elements that direct, filter or otherwise conduct emitted light so that it arrives at its intended target in a usable form, and a control system that starts and stops the production of light when necessary.

Photodynamic therapy may be carried out using certain compositions, such as ALA, in connection with illuminators as described above. Such compositions and/or devices are disclosed, for example, in (1) U.S. Pat. No. 5,954,703 to Golub, entitled “Method and apparatus for applying 5-aminolevulinic acid,” issued on Sep. 21, 1999, (2) U.S. Pat. No. 6,223,071 to Lundahl et al., entitled “Illuminator for photodynamic therapy and diagnosis which produces substantially uniform intensity visible light,” issued on Apr. 24, 2001, (3) U.S. Pat. No. 10,814,114 to Boyajian et al., entitled “Method And Apparatus For Applying A Topical Solution,” issued on Oct. 27, 2020, (4) International Application No. PCT/US2016/056572 to Boyajian et al., entitled “Adjustable Illuminator For Photodynamic Therapy And Diagnosis,” published on Apr. 20, 2017 as WO 2017/066270, (5) U.S. Pat. No. 10,589,122 to Boyajian et al., entitled “Adjustable Illuminator For Photodynamic Therapy And Diagnosis,” issued on Mar. 17, 2020, and (6) U.S. Pat. No. 10,603,508 to Boyajian et al., entitled “Adjustable Illuminators and Methods For Photodynamic Therapy and Diagnosis,” issued on Mar. 31, 2020.

However, one downside of PDT is that patients often experience pain during the illumination phase. Pain associated with PDT is the most severe adverse effect and may lead to interruption or discontinuation of treatment, resulting in refusal to repeat the process at a future date owing to unbearable discomfort. This pain is not alleviated by any topical anesthetic agent (other than ice chips and evaporative cooling), and can be so severe that patients refuse to complete the treatment and/or decline to undergo any future PDT (Warren CB et al. J Am Acad Dermatol 2009;61:1033-43; Ang JM et al. Photodiagnosis Photodyn Ther 2017;19:308-44).

Therefore, PDT-related pain represents a significant barrier to achieving optimal therapeutic outcomes. The lengthy procedure, wait time and associated pain has led more and more patients to opt out of the therapy. Pain is thought to be directly proportional to the length of pre-illumination incubation time as more photosensitizer is allowed to accumulate prior to the light exposure. There is a need in the art to decrease incubation times to shorten clinic visits and reduce associated pain, while maintaining comparable treatment outcomes.

### SUMMARY OF THE INVENTION

The inventors have surprisingly found that a topical composition for photodynamic therapy comprising at least one penetration enhancer and at least one chelating agent with a 5-aminoketone compound when applied into the skin of the patient showed an increase in penetration of 5-aminoketone compound to the skin and also an increase in conversion of this compound to PpIX in the skin. Further, the incubation time in the photodynamic therapy was reduced by at least 30% compared to a composition which is devoid of a penetration enhancer and/or a chelating agent. The chelating agent was found to enhance the accumulation of PpIX. Without being bound by any theory, it is believed that this accumulation of PpIX was enhanced by the chelation of iron, thereby preventing the inactivating action of ferrochelatase in incorporating the metal into PpIX, leading to PpIX build-up. The photosensitizing effect is thus enhanced.

The present invention is drawn to a topical composition for photodynamic therapy in the treatment of a dermatological disorder, comprising:
a) a 5-carbon aminoketone compound of Formula I

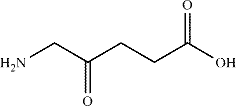

or its pharmaceutically acceptable salt
b) at least one penetration enhancer,
c) at least one chelating agent, and
d) optionally, an antifoaming agent.

In some embodiments, the present invention is drawn to a topical composition for the treatment of a dermatological disorder, comprising:
a) a 5-carbon aminoketone compound of Formula I

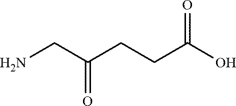

or its pharmaceutically acceptable salt, and
b) a vehicle, wherein the vehicle comprises:
   (i) at least one penetration enhancer, and
   (ii) at least one chelating agent,
wherein the topical composition is applied to the affected area of the patient (e.g., the skin) for a period in the range of 15 minutes to 10 hours.

In some preferable embodiments, the vehicle further comprises an optional antifoaming agent. In another preferable embodiment, the method of treatment is photodynamic therapy. In another preferable embodiment, after application of the composition, the affected area is illuminated with a light source. In another preferable embodiment, the compound of Formula I is in the form of a dry solid.

In some embodiments, the present invention is drawn to a topical composition for photodynamic therapy in the treatment of a dermatological disorder, comprising:
a) a 5-carbon aminoketone compound of Formula I

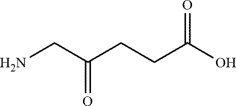

or its pharmaceutically acceptable salt, in the form of a dry solid, and
b) a vehicle, wherein the vehicle comprises
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area of the patient (e.g., the skin) with a light source.

In some embodiments, the at least one penetration enhancer is selected from a group consisting of dialkyl derivatives of acetamide and formamide, pyrrolidone derivatives, fatty acids, glycol derivatives, azones, polysorbates, macrogolglycerides, polyethylene glycol derivatives, ethoxylated ether derivatives, and glycosaminoglycan.

In some embodiments, the at least one chelating agent selected from a group consisting of ethylenediaminetetraacetic acid (EDTA) and its pharmaceutically acceptable salts like disodium edetate, disodium edetate dehydrate, di-potassium edetate, dipotassium edetate dehydrate, edetate calcium disodium, and diethylenetriamine pentaacetic acid.

The present invention is drawn to a topical composition for photodynamic therapy in the treatment of a dermatological disorder, comprising:
a) 5-ALA or its pharmaceutically acceptable salt,
b) propylene glycol,
c) EDTA or its pharmaceutically acceptable salt, and
d) optionally, an antifoaming agent.

In some embodiments, the present invention is drawn to a topical composition for photodynamic therapy in the treatment of a dermatological disorder, comprising:
a) 5-ALA or its pharmaceutically acceptable salt, in the form of a dry solid, and
b) a vehicle, wherein the vehicle comprises
   (i) propylene glycol,
   (ii) EDTA and pharmaceutically acceptable salts and
   (iii) optionally, an antifoaming agent.

The present invention is also drawn to a method for photodynamic therapy in the treatment of actinic keratosis, comprising administering a topical composition to the skin of the patient comprising:
a) a 5-carbon aminoketone compound of Formula I

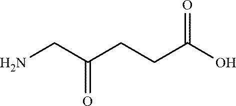

or its pharmaceutically acceptable salt
b) at least one penetration enhancer,
c) at least one chelating agent, and
d) optionally, an antifoaming agent.

In some embodiments, the present invention is also drawn to a method in the treatment of actinic keratosis comprising, administering a topical composition to the skin of the patient comprising:
a) a 5-carbon aminoketone compound of Formula I

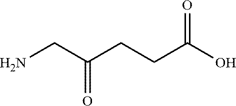

or its pharmaceutically acceptable salt, and
b) a vehicle, wherein the vehicle comprises:
   (i) at least one penetration enhancer, and
   (ii) at least one chelating agent,
wherein the topical composition is applied for a period in the range of 15 minutes to 10 hours, followed by illuminating the affected area of the patient with a light source.

In a preferable embodiment, the method comprises the vehicle comprising an optional antifoaming agent. In another preferable embodiment, the method of treatment is photodynamic therapy. In another preferable embodiment, the compound of Formula I is in the form of a dry solid.

In some embodiments, the present invention is also drawn to a method for photodynamic therapy in the treatment of actinic keratosis comprising administering a topical composition to the skin of the patient comprising:
a) a 5-carbon aminoketone compound of Formula I

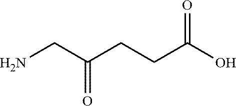

or its pharmaceutically acceptable salt in the form of a dry solid, and
b) a vehicle, wherein the vehicle comprises
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area with a light source.

### BRIEF DESCRIPTION OF DRAWINGS

FIG. 1 - illustrates a test set up of an in-vitro skin model used in Examples 2-5.

FIG. 2 - is a graph showing a comparison of the amount of PpIX in receptor solution for illustrative formulations of the invention compared to a Reference Listed Drug (“RLD”).

FIG. 3 - is a graph showing skin retention information of PpIX for illustrative formulations of the invention compared to RLD.

FIG. 4 - is a graph showing a comparison of the amount of PpIX in receptor solution and skin retention for formulations comprising EDTA versus formulations comprising edetate calcium disodium.

FIG. 5 - is a graph showing the effect of temperature on PpIX formulation by determining the amount of PpIX in receptor solution.

FIG. 6 - Permeation data summary for Donor 1 (A), Donor 2 (B) and Donor 3 (C), blank data was subtracted from the results.

FIG. 7 - is a graph showing PpIX content quantified inside the skin compartment for Donors 1, 2, and 3.

FIG. 8 - is a graph showing the effect of PG and EDTA on PpIX levels in the receptor compartment (A) and in the skin (B) in three donors (Donors 1, 2 and 3).

FIG. 9 - is graphs showing synergetic effect of Transcutol®, PG and EDTA on PpIX levels in the receptor compartment (A) and in the skin (B) in three donors (Donors 1, 2 and 3).

### DETAILED DESCRIPTION OF THE INVENTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The following terms are used throughout and are as defined below.

As used herein and in the appended claims, singular articles such as “a” and “an” and “the” and similar references in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illustrate the embodiments and does not pose a limitation on the scope of the claims unless otherwise stated. No language in the specification should be construed as indicating any non-claimed element as essential.

The embodiments, illustratively described herein may suitably be practiced in the absence of any element or elements, and limitation or limitations. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The expression “comprising” means “including, but not limited to.” Thus, other non-mentioned substances, additives, devices or steps may be present. Unless otherwise specified, “a” or “an” means one or more.

Unless otherwise indicated, all numbers expressing quantities of properties, parameters, conditions, and so forth, used in the specification and claims are to be understood as being modified in all instances by the term “about.” Accordingly, unless indicated to the contrary, the numerical parameters set forth in the following specification and attached claims are approximations. Any numerical parameter should at least be construed in light of the number of significant digits and by applying ordinary rounding techniques. The term “about” when used before a numerical designation, e.g., temperature, time, amount, and concentration, including range, indicates approximations which may vary by (+) or (-) 10%, 5% or 1%.

As will be understood by one of skill in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

The term ‘synergistic’ as used herein is defined to mean a combination of components wherein the activity of the combination is greater than the additive of the individual activities of each component of the combination.

According to one embodiment, the present invention provides a topical composition for photodynamic therapy in the treatment of a dermatological disorder, comprising:
a) a 5-carbon aminoketone compound of Formula I

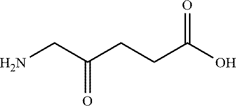

or its pharmaceutically acceptable salt
b) at least one penetration enhancer,
c) at least one chelating agent, and
d) optionally, an antifoaming agent.

Preferably, the at least one penetration enhancer is selected from a group consisting of dialkyl derivatives of acetamide and formamide, pyrrolidone derivatives, fatty acids, fatty acid esters, glycol derivatives, glycerides, azones, polysorbates, macrogolglycerides, polyethylene glycol derivatives, ethoxylated ether derivatives, bile salts and glycosaminoglycan. More preferably, the topical compositions of the present invention comprises dialkyl derivatives of acetamide and formamide such as dimethyl acetamide, dimethyl formamide, pyrrolidone derivatives such as N-methyl-2- Pyrrolidone, fatty acids such as oleic acid, glycol derivatives such as propylene glycol and its fatty esters such as propylene glycol monocaprylate, propylene glycol monolaurate, azones such as laurocapram or 1-n-dodecyl-azacycloheptan-2-one, polysorbates, such as Tween 80, macrogolglycerides such as stearoyl macrogolglycerides, oleoyl macrogolglycerides, lauroyi macrogolglycerides, capryl-caproyl macrogolglycerides, polyethylene glycol derivatives such as polyethylene glycol 400, ethoxylated ether derivatives such as diethyleneglycol monoethyl, diethyleneglycol monomethyl ether; and dipropyleneglycol monomethyl ether, glycosaminoglycan such as chondroitin sulfate, keratan sulfate, dermatan sulfide, heparin sulphate and heparan sulphate as the permeation enhancer.

The penetration enhancer is present in the composition in an amount in the range of about 10% w/w to about 50% w/w of the composition, including for example, about 10%, 20%, 30%, 40%, or 50% w/w of the composition and any and all ranges and subranges therein. More preferably, the penetration enhancer is present in the composition in an amount in the range of about 20% w/w to about 40% w/w of the composition, including for example, about 20%, about 30%, or about 40% w/w of the composition and any and all ranges and subranges therein.

In a preferred embodiment, the at least one penetration enhancer is selected from a group consisting of glycol derivatives, polyethylene glycol derivatives, and ethoxylated ether derivatives. In another preferred embodiment, the at least one penetration enhancer is selected from a group consisting of propylene glycol, polyethylene glycol, and 2-(2-Ethoxyethoxy)ethanol (Transcutol®). Propylene glycol is present in the composition in an amount in the range of about 10% w/w to about 50% w/w of the composition, including for example, about 10%, 20%, 30%, 40%, or 50% w/w of the composition and any and all ranges and subranges therein. Preferably, propylene glycol is present in the composition in an amount in the range of about 20% w/w to about 40% w/w of the composition, including for example, about 20%, about 30%, or about 40% w/w of the composition and any and all ranges and subranges therein. 2-(2-Ethoxyethoxy)ethanol (Transcutol®) when used as a penetration enhancer is present in the composition in an amount in the range of about 2% w/w to about 50% w/w of the composition, including for example, about 2%, 4%, 6%, 8%, 10%, 12%, 14%, 16%, 18%, 20%, 22%, 24%, 26%, 28%, 30%, 32%, 34%, 36%, 38%, or 40% w/w of the composition and any and all ranges and subranges therein. Preferably, 2-(2-Ethoxyethoxy)ethanol is present in the composition in an amount in the range of about 4% w/w to about 10% w/w of the composition, including for example, about 4%, 5%, 6%, 7%, 8%, 9% or 10% w/w of the composition and any and all ranges and subranges therein.

In another embodiment, the at least one chelating agent selected from a group consisting of ethylenediaminetetraacetic acid (EDTA) and its pharmaceutically acceptable salts like disodium edetate, disodium edetate dehydrate, trisodium edetate, di-potassium edetate, dipotassium edetate dehydrate, edetate calcium disodium, diethylenetriamine pentaacetic acid, and organic acid such as citric acid, fumaric acid, malic acid, lactic acid and glycolic acid. Preferably, the at least one chelating agent is disodium edetate.

The at least one chelating agent may be present in the composition in an amount in the range of about 0.01% w/w to about 2% w/w of the composition, including for example, about 0.01%, 0.05%, 0.1%, 0.2%, 0.25%, 0.4%, 0.5%, 0.75%, 0.80%, 0.90%, 1.0%, 1.1%, 1.2%, 1.25%, 1.4%, 1.5%, 1.75%, 1.80%, 1.90% or 2.0% w/w of the composition and any and all ranges and subranges therein. Preferably, an amount in the range of about 0.05% w/w to about 1% w/w, including for example, about 0.05%, 0.1%, 0.2%, 0.25%, 0.4%, 0.5%, 0.75%, 0.80%, 0.90% or 1.0% w/w of the composition and any and all ranges and subranges therein.

EDTA or its pharmaceutically acceptable salt when used as a chelating agent may be present in the composition in an amount in the range of about 0.01% w/w to about 2% w/w of the composition, including for example, about 0.01%, 0.05%, 0.1%, 0.2%, 0.25%, 0.4%, 0.5%, 0.75%, 0.80%, 0.90%, 1.0%, 1.1%, 1.2%, 1.25%, 1.4%, 1.5%, 1.75%, 1.80%, 1.90% or 2.0% w/w of the composition and any and all ranges and subranges therein. Preferably, an amount in the range of about 0.05% w/w to 1% w/w, including for example, about 0.05%, 0.1%, 0.2%, 0.25%, 0.4%, 0.5%, 0.75%, 0.80%, 0.90% or 1.0% w/w of the composition and any and all ranges and subranges therein. In a most preferred embodiment, EDTA or its pharmaceutically acceptable salt is present in the composition in an amount of about 0.1% w/w to about 0.25% w/w of the composition.

It was surprisingly found that the combination of a chelating agent and permeation enhancer showed synergistic effect on an increase in the permeation of 5-ALA and PpIX formation. Preferably, the combination of propylene glycol and EDTA or its salts showed synergistic effect on an increase in the permeation of 5-ALA and PpIX formation. Addition of Transcutol® to the combination of a chelating agent and permeation enhancer further enhance the synergy effect of the combination. Preferably, addition of Transcutol® to the combination of propylene glycol and EDTA or its salts, showed a synergistic effect on the increase in the permeation of 5-ALA and PpIX formation.

In another embodiment, the combination of a chelating agent and permeation enhancer showed an additive effect on increase in the permeation of 5-ALA and PpIX formation. Preferably, the combination of propylene glycol and EDTA or its salts showed additive effect on an increase in the permeation of 5-ALA and PpIX formation. Addition of Transcutol® to the combination of a chelating agent and permeation enhancer further enhance the synergy effect of the combination. For example, addition of Transcutol® to the combination of propylene glycol and EDTA or its salts, showed an additive effect on the increase in the permeation of 5-ALA and PpIX formation.

In one embodiment, the 5-carbon aminoketone compound is 5-aminolevulinic acid (ALA) or its pharmaceutically acceptable salt. Preferably, the 5-carbon aminoketone compound is a hydrochloride salt of aminolevulinic acid.

The compound of Formula I

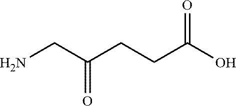

or its pharmaceutically acceptable salt is present in the composition in an amount in the range of about 10% w/w to70% w/w of the composition, including for example, about 10%, 20%, 30%, 40%, 50%, 60% or 70% w/w of the composition. Preferably in an amount in the range about 20% w/w to 50% w/w of the composition, including for example, about 20%, 25%, 30%, 35%, 40%, 45% or 50% w/w of the composition. In a most preferred embodiment, the compound of Formula I or its pharmaceutically acceptable salt is present in an amount of about 20 % w/w.

The compound of 5-ALA or its pharmaceutically acceptable salt is present in the composition in an amount in the range of about 10% w/w to about 70% w/w of the composition, including for example, about 10%, 20%, 30%, 40%, 50%, 60% or 70% w/w of the composition and any and all ranges and subranges therein.. Preferably in an amount in the range of about 20% w/w to about 50% w/w of the composition including for example, about 20%, 25%, 30%, 35%, 40%, 45% or 50% w/w of the composition and any and all ranges and subranges therein. In a most preferred embodiment, 5-ALA or its pharmaceutically acceptable salt is present in the composition in an amount of about 20 % w/w.

The compositions of the present invention may contain a variety of other inactive ingredients that are conventionally used in given product types provided that they do not unacceptably alter the benefits of the invention. The inactive ingredients may be selected from alcohol, isopropyl alcohol, polyethylene glycol, propylene glycol, glycerine, diethylene glycol monoethyl ether or purified water or combinations thereof. It may further comprise a surfactant or a wetting agent and/or a humectant. The surfactant or wetting agent may be selected from the group consisting of laureth-4, sodium lauryl sulphate, sodium dodecyl sulfate, ammonium lauryl sulphate or sodium octech-1/deceth-1 sulfate thereof. The humectant may be selected from the group consisting of polyethylene glycol, propylene glycol, hyaluronic acid or glycerine thereof.

In one embodiment, the topical compositions of the present invention optionally comprise an anti-foaming agent. Suitable anti-foaming agents may include, but are not limited to polydimethylsiloxanes and other silicones, certain alcohols, stearates and glycols. Preferably, the anti-foaming agent is cyclic polydimethylsiloxane. More preferably, the anti-foaming agent is cyclomethicone. The anti-foaming agent is present in the composition in an amount in the range of about 0.2% w/w to about 1.0% w/w of the composition, including for example about 0.2%, 0.25%, 0.4%, 0.5%, 0.75%, 0.80%, 0.90%, or 1.0% w/w of the composition and any and all ranges and subranges therein. Preferably, the anti-foaming agent is present in the composition in an amount in the range of about 0.2% w/w to about 0.5% w/w of the composition. More preferably, the anti-foaming agent is present in the composition in an amount of about 0.5% w/w of the composition.

The composition may be prepared by simple admixture of ALA with the vehicle. The vehicle may be prepared by mixing of permeation enhancer, chelating agent, anti-foaming agent and other inactive ingredients in any order. Preferably, the vehicle may be prepared by adding the ingredients in the following order, to purified water, chelating agent added and mixed well. To this, solvent such as ethyl alcohol was added, then propylene glycol was added, then polyethylene glycol 400, then isopropyl alcohol, then Transcutol®, and then Laureth-4 was added. The vehicle was mixed after addition of each ingredient. Finally, anti-foaming agent such as cyclomethicone was added to get the final mix. It has been surprisingly discovered that the order of addition in this order prevents precipitation of the chelating agent, such as EDTA.

In one embodiment, the present invention is drawn to a topical composition for the treatment of a dermatological disorder, comprising:
a) a 5-carbon aminoketone compound of Formula I

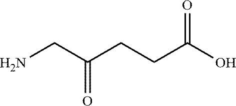

or its pharmaceutically acceptable salt, and
b) a vehicle, wherein the vehicle comprises:
   (i) at least one penetration enhancer, and
   (ii) at least one chelating agent,
wherein the topical composition is applied to the affected area of the skin for a period in the range of 15 minutes to 10 hours.

In a preferable embodiment, the vehicle comprises an optional antifoaming agent. In another preferable embodiment, the method of treatment is photodynamic therapy. In another preferable embodiment, after application of the composition, the affected area is illuminated with a light source. In another preferable embodiment, the composition comprises the compound of Formula I in the form of a dry solid.

In another embodiment, the present invention is drawn to a topical composition for use in a photodynamic therapy for the treatment of a dermatological disorder, comprising:
a) a 5-carbon aminoketone compound of Formula I

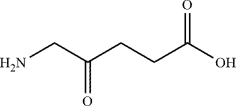

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area of the skin with a light source.

It was surprisingly found that the topical composition of the invention reduces the incubation time in the photodynamic therapy by at least about 30% compared to a composition which is devoid of a penetration enhancer and chelating agent. Preferably, the incubation time in the photodynamic therapy is reduced by at least about 30% compared to a composition which is devoid of a penetration enhancer and chelating agent. Preferably, the incubation time in the photodynamic therapy is reduced by at least about 40% compared to a composition which is devoid of a penetration enhancer and chelating agent. Preferably, the incubation time in the photodynamic therapy is reduced by at least about 50% compared to a composition which is devoid of a penetration enhancer and chelating agent. Preferably, the incubation time in the photodynamic therapy is reduced by at least about 60% compared to a composition which is devoid of a penetration enhancer and chelating agent. Preferably, the incubation time in the photodynamic therapy is reduced by at least about 70% compared to a composition which is devoid of a penetration enhancer and chelating agent. Preferably, the incubation time in the photodynamic therapy is reduced by at least about 80% compared to a composition which is devoid of a penetration enhancer and chelating agent. Preferably, the incubation time in the photodynamic therapy is reduced by at least about 90% compared to a composition which is devoid of a penetration enhancer and chelating agent. Preferably, the incubation time in the photodynamic therapy is reduced by at least about 95% compared to a composition which is devoid of a penetration enhancer and chelating agent.

Such reductions in incubation time are believed to reduce pain associated with PDT.

In another embodiment, the present invention provides a topical composition for photodynamic therapy in the treatment of a dermatological disorder, comprising:
a) a 5-carbon aminoketone compound of Formula I

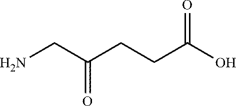

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area of the patient (e.g., the skin) with a light source.

The topical composition is applied for a period of, for example, 15 minutes, 2 hours, 4 hours, 6 hours, 8 hours, or 10 hours.

In another embodiment, the present invention provides a topical composition for use in a photodynamic therapy for the treatment of a dermatological disorder, comprising:
a) a 5-carbon aminoketone compound of Formula I

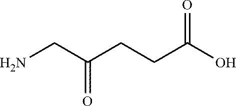

or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, ethoxylated ether derivatives,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area of the patient with a light source.

In another embodiment, the present invention provides a topical composition for use in a photodynamic therapy for the treatment of a dermatological disorder, comprising:
a) 5-ALA or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area of the patient with a light source.

In another embodiment, the present invention provides a topical composition for use in a photodynamic therapy for the treatment of a dermatological disorder, comprising:
a) a 5-carbon aminoketone compound of Formula I

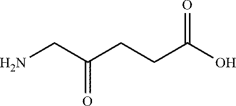

or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area of the patient with a light source.

In another embodiment, the present invention provides a topical composition for use in a photodynamic therapy for the treatment of a dermatological disorder, comprising:
a) 5-ALA or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area of the skin with a light source.

In another embodiment, the present invention provides a topical composition for photodynamic therapy in the treatment of a dermatological disorder, comprising:
a) 5-ALA or its pharmaceutically acceptable salt,
b) propylene glycol,
c) EDTA or its pharmaceutically acceptable salt, and
d) optionally, an antifoaming agent.

In yet another embodiment, the present invention provides a topical composition for photodynamic therapy in the treatment of a dermatological disorder, comprising:
a) 5-ALA or its pharmaceutically acceptable salt, in the form of a dry solid, and
b) a vehicle, wherein the vehicle comprises:
   (i) propylene glycol,
   (ii) EDTA and pharmaceutically acceptable salts, and
   (iii) optionally, an antifoaming agent.

In yet another embodiment, the present invention provides a topical composition for use in a photodynamic therapy for the treatment of a dermatological disorder, comprising:
a) a 5-carbon aminoketone compound of Formula I

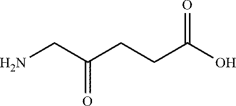

or its pharmaceutically acceptable salt in the form of a dry solid, and
b) a vehicle, wherein the vehicle comprises:
   (i) propylene glycol,
   (ii) 2-(2-Ethoxyethoxy)ethanol, and
   (iii) disodium edetate, and
   (iv) optionally, an antifoaming agent.

In yet another embodiment, the present invention provides a topical composition for use in a photodynamic therapy for the treatment of a dermatological disorder, comprising:
a) 5-ALA or its pharmaceutically acceptable salt in the form of a dry solid, and
b) a vehicle, wherein the vehicle comprises:
   (i) propylene glycol,
   (ii) 2-(2-Ethoxyethoxy)ethanol, and
   (iii) disodium edetate, and
   (iv) optionally, an antifoaming agent.

In yet another embodiment, the present invention provides a topical composition for use in a photodynamic therapy for the treatment of a dermatological disorder, comprising:
a) a 5-carbon aminoketone compound of Formula I

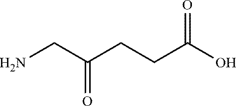

or its pharmaceutically acceptable salt in the form of a dry solid, and
b) a vehicle, wherein the vehicle comprises:
   (i) propylene glycol in an amount in the range of about 10% w/w to about 50% w/w,
   (ii) 2-(2-Ethoxyethoxy)ethanol in an amount in the range of about 2% w/w to about 50% w/w, and (iii) disodium edetate, and
   (iv) optionally, an antifoaming agent.

In yet another embodiment, the present invention provides a topical composition for use in a photodynamic therapy for the treatment of a dermatological disorder, comprising:
a) 5-ALA or its pharmaceutically acceptable salt in the form of a dry solid, and
b) a vehicle, wherein the vehicle comprises:
   (i) propylene glycol in an amount in the range of about 10% w/w to about 50% w/w,
   (ii) 2-(2-Ethoxyethoxy)ethanol in an amount in the range of about 2% w/w to about 50% w/w, and
   (iii) disodium edetate, and
   (iv) optionally, an antifoaming agent.

In another preferred embodiment, the present invention provides a topical composition for use in a photodynamic therapy for the treatment of a dermatological disorder, comprising:
a) a 5-carbon aminoketone compound of Formula I or its pharmaceutically acceptable salt,

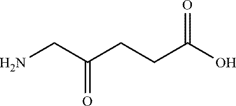

b) propylene glycol,
c) 2-(2-Ethoxyethoxy)ethanol, and
d) disodium edetate.

In another preferred embodiment, the present invention provides a topical composition for use in a photodynamic therapy for the treatment of a dermatological disorder, comprising:
a) 5-aminolevulinic acid in an amount of 20% w/w of the composition,
b) propylene glycol in an amount of 20% to 40% w/w of the composition, and
c) EDTA in an amount of 0.1% to 0.5% w/w of the composition.

Preferably, the composition further comprises 2-(2-Ethoxyethoxy)ethanol in an amount of 4% to 10% w/w of the composition.

In another preferred embodiment, the composition further comprises cyclomethicone in an amount of 0.2 to 0.5% w/w of the composition.

In a more preferred embodiment, the present invention provides a topical composition for use in a photodynamic therapy for the treatment of a dermatological disorder, comprising:
a) a 5-carbon aminoketone compound of Formula I

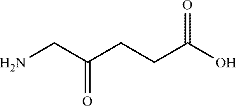

or its pharmaceutically acceptable salt, in an amount of 1-30% w/w,
b) propylene glycol in an amount in the range of about 10% w/w to about 50% w/w,
c) 2-(2-Ethoxyethoxy)ethanol in an amount in the range of about 2% w/w to about 50% w/w, and
d) disodium edetate.

In another embodiment, the present invention provides a topical composition for use in a photodynamic therapy for the treatment of a dermatological disorder, comprising:
a) a 5-carbon aminoketone compound of Formula I

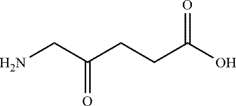

or its pharmaceutically acceptable salt,
b) ethanol,
c) Laureth-4
d) polyethylene glycol,
e) isopropyl alcohol,
f) propylene glycol,
g) 2-(2-Ethoxyethoxy)ethanol,
h) edetate disodium,
i) cyclomethicone, and
j) purified water.

In another embodiment, the present invention provides a topical composition for use in a photodynamic therapy for the treatment of a dermatological disorder, comprising:
a) a 5-carbon aminoketone compound of Formula I

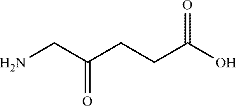

or its pharmaceutically acceptable salt in the form of a dry solid, and
b) a vehicle, wherein the vehicle comprises:
   (i) ethanol,
   (ii) Laureth-4,
   (iii) polyethylene glycol,
   (iv) isopropyl alcohol,
   (v) propylene glycol,
   (vi) 2-(2-Ethoxyethoxy)ethanol,
   (vii) edetate disodium,
   (viii) cyclomethicone, and
   (ix) purified water.

In another embodiment, the present invention provides a topical composition for use in a photodynamic therapy for the treatment of a dermatological disorder, comprising:
a) 5-ALA or its pharmaceutically acceptable salt,
b) ethanol,
c) Laureth-4,
d) polyethylene glycol,
e) isopropyl alcohol,
f) propylene glycol,
g) 2-(2-Ethoxyethoxy)ethanol,
h) edetate disodium,
i) cyclomethicone, and
j) purified water.

In another embodiment, the present invention provides a topical composition for use in a photodynamic therapy for the treatment of a dermatological disorder, comprising:
a) 5-ALA or its pharmaceutically acceptable salt in the form of a dry solid, and
b) a vehicle, wherein the vehicle comprises:
   (i) ethanol,
   (ii) Laureth-4,
   (iii) polyethylene glycol,
   (iv) isopropyl alcohol,
   (v) propylene glycol,
   (vi) 2-(2-Ethoxyethoxy)ethanol,
   (vii) edetate disodium,
   (viii) cyclomethicone, and
   (ix) purified water.

In a most preferred embodiment, the present invention provides a topical composition for use in photodynamic therapy for the treatment of a dermatological disorder, comprising:
a) a 5-carbon aminoketone compound of Formula I or its pharmaceutically acceptable salt in an amount of 20% w/w,

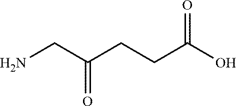

b) ethanol in an amount of 10 to 15% w/w of the composition,
c) Laureth-4 in an amount of 5 to 10% w/w of the composition,
d) polyethylene glycol in an amount of 1 to 5% w/w of the composition,
e) isopropyl alcohol in an amount of 2 to 4% w/w of the composition,
f) propylene glycol in an amount of 20 to 40% w/w of the composition,
g) 2-(2-Ethoxyethoxy)ethanol in an amount of 2 to 4% w/w of the composition,
h) edetate disodium in an amount of 0.1 to 0.25% w/w of the composition,
i) cyclomethicone in an amount of 0.2 to 0.5% w/w of the composition, and
j) purified water.

In another preferred embodiment, the present invention provides a topical composition for use in a photodynamic therapy for the treatment of a dermatological disorder, comprising:
a) 5-ALA or its pharmaceutically acceptable salt in an amount of 20% w/w,
b) ethanol in an amount of 10 to 15% w/w of the composition,
c) Laureth-4 in an amount of 5 to 10% w/w of the composition,
d) polyethylene glycol in an amount of 1 to 5% w/w of the composition,
e) isopropyl alcohol in an amount of 2 to 4% w/w of the composition,
f) propylene glycol in an amount of 20 to 40 % w/w of the composition,
g) 2-(2-Ethoxyethoxy)ethanol in an amount of 2 to 4% w/w of the composition,
h) edetate disodium in an amount of 0.1 to 0.25% w/w of the composition,
i) cyclomethicone in an amount of 0.2 to 0.5% w/w of the composition, and
j) purified water.

In yet another embodiment, the present invention provides a topical composition for use in a photodynamic therapy for the treatment of a dermatological disorder, comprising:
a) a 5-carbon aminoketone compound of Formula I

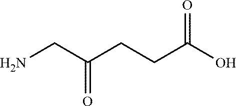

or its pharmaceutically acceptable salt in the form of a dry solid, and
b) a vehicle, wherein the vehicle comprises:
   (i) ethanol in an amount of 10 to 15% w/w of the composition,
   (ii) Laureth-4 in an amount of 5 to 10% w/w of the composition,
   (iii) polyethylene glycol in an amount of 1 to 5% w/w of the composition,
   (iv) isopropyl alcohol in an amount of 2 to 4% w/w of the composition,
   (v) propylene glycol in an amount of 20 to 40% w/w of the composition,
   (vi) 2-(2-Ethoxyethoxy)ethanol in an amount of 2 to 4% w/w of the composition,
   (vii) edetate disodium in an amount of 0.1 to 0.25% w/w of the composition, and
   (viii) cyclomethicone in an amount of 0.2 to 0.5% w/w of the composition.

In another embodiment, the present invention is drawn to a method for photodynamic therapy in the treatment of actinic keratosis, comprising administering a topical composition to the skin of a patient comprising:
a) a 5-carbon aminoketone compound of Formula I

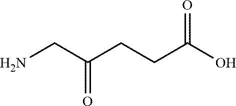

or its pharmaceutically acceptable salt,
b) at least one penetration enhancer,
c) at least one chelating agent, and
d) optionally, an antifoaming agent.

In another embodiment, the present invention is drawn to a method in the treatment of actinic keratosis comprising, administering a topical composition to the skin of a patient comprising:
a) a 5-carbon aminoketone compound of Formula I

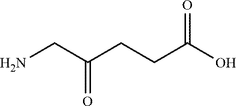

or its pharmaceutically acceptable salt, and
b) a vehicle, wherein the vehicle comprises:
   (i) at least one penetration enhancer, and
   (ii) at least one chelating agent,
wherein the topical composition is applied for a period in the range of 15 minutes to 10 hours, followed by illuminating the affected area of the patient with a light source.

In a preferable embodiment, the method comprises the vehicle comprising an optional antifoaming agent. In another preferable embodiment, the method of treatment is photodynamic therapy. In another preferable embodiment, the compound of Formula I is in the form of a dry solid.

In yet some embodiments, the present invention is also drawn to a method for photodynamic therapy in the treatment of actinic keratosis, comprising administering a topical composition to the skin of a patient comprising:
a) a 5-carbon aminoketone compound of Formula I

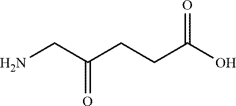

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area with a light source.

In one embodiment, the method for photodynamic therapy in the treatment of actinic keratosis, comprises application of topical composition for a period of, for example, 15 minutes, 2 hours, 4 hours, 6 hours, 8 hours, or 10 hours.

In yet another embodiment, the present invention provides a method for photodynamic therapy in the treatment of actinic keratosis, comprising administering a topical composition to the skin of a patient comprising:
a) a 5-carbon aminoketone compound of Formula I

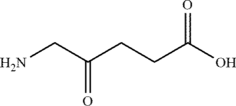

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area with a light source.

In yet another embodiment, the present invention provides a method for photodynamic therapy in the treatment of actinic keratosis, comprising administering a topical composition to the skin of a patient comprising:
a) a 5-carbon aminoketone compound of Formula I

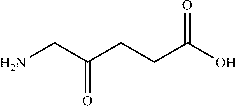

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein the topical composition is applied for a period in the range of 15 minutes to 10 hours, followed by illuminating the affected area with a light source.

In yet another embodiment, the present invention provides a method for photodynamic therapy in the treatment of actinic keratosis, comprising administering a topical composition to the skin of the patient comprising:
a) a 5-carbon aminoketone compound of Formula I

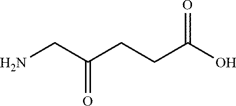

or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area with a light source.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area with a light source.

In yet another embodiment, the present invention provides a method for photodynamic therapy in the treatment of actinic keratosis, comprising administering a topical composition to the skin of a patient comprising:
a) a 5-carbon aminoketone compound of Formula I

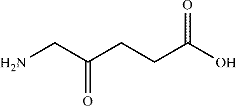

or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent,
wherein the topical composition is applied for a period in the range of 15 minutes to 10 hours, followed by illuminating the affected area with a light source.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent.
wherein the topical composition is applied for a period in the range of 15 minutes to 10 hours, followed by illuminating the affected area with a light source.

In yet another embodiment, the present invention provides a method for photodynamic therapy in the treatment of actinic keratosis, comprising administering a topical composition to the skin of a patient comprising:
a) a 5-carbon aminoketone compound of Formula I

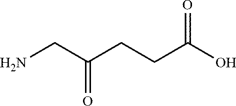

or its pharmaceutically acceptable salt in the form of a dry solid, and
b) a vehicle, wherein the vehicle comprises:
   (i) propylene glycol,
   (ii) 2-(2-Ethoxyethoxy)ethanol, and
   (iii) disodium edetate, and
   (iv) optionally, an antifoaming agent.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) propylene glycol,
   (ii) 2-(2-Ethoxyethoxy)ethanol, and
   (iii) disodium edetate, and
   (iv) optionally, an antifoaming agent.

In yet another embodiment, the present invention provides a method for photodynamic therapy in the treatment of actinic keratosis, comprising administering a topical composition to the skin of a patient comprising:
a) a 5-carbon aminoketone compound of Formula I

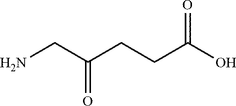

or its pharmaceutically acceptable salt in the form of a dry solid, and
b) a vehicle, wherein the vehicle comprises:
   (i) propylene glycol in an amount in the range of about 10% w/w to about 50% w/w,
   (ii) 2-(2-Ethoxyethoxy)ethanol in an amount in the range of about 2% w/w to about 50% w/w, and
   (iii) disodium edetate, and
   (iv) optionally, an antifoaming agent.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) propylene glycol in an amount in the range of about 10% w/w to about 50% w/w,
   (ii) 2-(2-Ethoxyethoxy)ethanol in an amount in the range of about 2% w/w to about 50% w/w, and
   (iii) disodium edetate, and
   (iv) optionally, an antifoaming agent.

In yet another embodiment, the present invention provides a method for photodynamic therapy in the treatment of actinic keratosis, comprising administering a topical composition to the skin of a patient comprising:
a) a 5-carbon aminoketone compound of Formula I or its pharmaceutically acceptable salt,

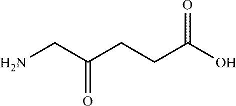

b) propylene glycol,
c) 2-(2-Ethoxyethoxy)ethanol, and
d) disodium edetate.

In yet another embodiment, the present invention provides a method for photodynamic therapy in the treatment of actinic keratosis, comprising administering a topical composition to the skin of a patient comprising:
a) a 5-carbon aminoketone compound of Formula I

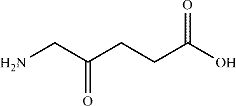

or its pharmaceutically acceptable salt, in an amount of 1-30% w/w,
b) propylene glycol in an amount in the range of about 10% w/w to about 50% w/w,
c) 2-(2-Ethoxyethoxy)ethanol in an amount of about 2% w/w to about 50% w/w, and
d) disodium edetate.

In yet another embodiment, the present invention provides a method for photodynamic therapy in the treatment of actinic keratosis, comprising administering a topical composition to the skin of a patient comprising:
a) a 5-carbon aminoketone compound of Formula I

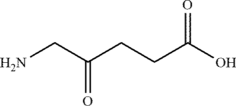

or its pharmaceutically acceptable salt,
b) ethanol,
c) Laureth-4,
d) polyethylene glycol,
e) isopropyl alcohol,
f) propylene glycol,
g) 2-(2-Ethoxyethoxy)ethanol,
h) edetate disodium,
i) cyclomethicone, and
j) purified water.

In yet another embodiment, the present invention provides a method for photodynamic therapy in the treatment of actinic keratosis, comprising administering a topical composition to the skin of a patient comprising:
a) a 5-carbon aminoketone compound of Formula I

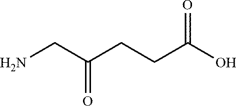

or its pharmaceutically acceptable salt in the form of a dry solid, and
b) a vehicle, wherein the vehicle comprises:
   (i) ethanol,
   (ii) Laureth-4,
   (iii) polyethylene glycol,
   (iv) isopropyl alcohol,
   (v) propylene glycol,
   (vi) 2-(2-Ethoxyethoxy)ethanol,
   (vii) edetate disodium,
   (viii) cyclomethicone, and
   (ix) purified water.

In yet another embodiment, the present invention provides a method for photodynamic therapy in the treatment of actinic keratosis, comprising administering a topical composition to the skin of a patient comprising:
a) 5-ALA or its pharmaceutically acceptable salt,
b) ethanol,
c) Laureth-4,
d) polyethylene glycol,
e) isopropyl alcohol,
f) propylene glycol,
g) 2-(2-Ethoxyethoxy)ethanol,
h) edetate disodium,
i) cyclomethicone, and
j) purified water.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) ethanol,
   (ii) Laureth-4,
   (iii) polyethylene glycol,
   (iv) isopropyl alcohol,
   (v) propylene glycol,
   (vi) 2-(2-Ethoxyethoxy)ethanol,
   (vii) edetate disodium,
   (viii) cyclomethicone, and
   (ix) purified water.

In yet another embodiment, the present invention provides a method for photodynamic therapy in the treatment of actinic keratosis, comprising administering a topical composition to the skin of a patient comprising:
a) a 5-carbon aminoketone compound of Formula I or its pharmaceutically acceptable salt in an amount of 20% w/w of the composition,

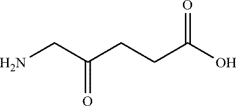

b) ethanol in an amount of 10 to 15% w/w of the composition,
c) Laureth-4 in an amount of 5 to 10% w/w of the composition,
d) polyethylene glycol in an amount of 1 to 5% w/w of the composition,
e) isopropyl alcohol in an amount of 2 to 4% w/w of the composition,
f) propylene glycol in an amount of 20 to 40% w/w of the composition,
g) 2-(2-Ethoxyethoxy)ethanol in an amount of 2 to 4% w/w of the composition,
h) edetate disodium in an amount of 0.1 to 0.25% w/w of the composition,
i) cyclomethicone in an amount of 0.2 to 0.5% w/w of the composition, and
j) purified water.

In yet another embodiment, the present invention provides a method for photodynamic therapy in the treatment of actinic keratosis, comprising administering a topical composition to the skin of a patient comprising:
a) 5-ALA or its pharmaceutically acceptable salt in an amount of 20% w/w of the composition,
b) ethanol in an amount of 10 to 15% w/w of the composition,
c) Laureth-4 in an amount of 5 to 10% w/w of the composition,
d) polyethylene glycol in an amount of 1 to 5% w/w of the composition,
e) Isopropyl alcohol in an amount of 2 to 4% w/w of the composition,
f) propylene glycol in an amount of 20 to 40% w/w of the composition,
g) 2-(2-Ethoxyethoxy)ethanol in an amount of 2 to 4% w/w of the composition,
h) edetate disodium in an amount of 0.1 to 0.25% w/w of the composition,
i) cyclomethicone in an amount of 0.2 to 0.5% w/w of the composition, and
j) purified water.

In yet another embodiment, the present invention provides a method for the treatment of actinic keratoses in a photodynamic therapy, comprising administering a topical composition to the skin of a patient comprising:
a) a 5-carbon aminoketone compound of Formula I

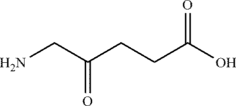

or its pharmaceutically acceptable salt in the form of a dry solid, and
b) a vehicle, wherein the vehicle comprises:
   (i) ethanol in an amount of 10 to 15% w/w of the composition,
   (ii) Laureth-4 in an amount of 5 to 10% w/w of the composition,
   (iii) polyethylene glycol in an amount of 1 to 5% w/w of the composition,
   (iv) isopropyl alcohol in an amount of 2 to 4 % w/w of the composition,
   (v) propylene glycol in an amount of 20 to 40% w/w of the composition,
   (vi) 2-(2-Ethoxyethoxy)ethanol in an amount of 2 to 4% w/w of the composition,
   (vii) edetate disodium in an amount of 0.1 to 0.25% w/w of the composition,
   (viii) cyclomethicone in an amount of 0.2 to 0.5% w/w of the composition, and
   (ix) purified water.

In yet another embodiment, the present invention provides a method for photodynamic therapy in the treatment of actinic keratosis, comprising administering a topical composition to the skin of a patient comprising:
a) a 5-carbon aminoketone compound of Formula I

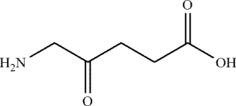

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein the method increases the penetration of 5-carbon aminoketone in the skin of a patient by at least about 30% when compared to the administration of a composition which is devoid of a penetration enhancer and chelating agent.

Preferably, the penetration of 5-carbon aminoketone in the skin of a patient in the photodynamic therapy is increased by at least about 30% compared to a composition which is devoid of a penetration enhancer and chelating agent. Preferably, the penetration of 5-carbon aminoketone in the skin of a patient in the photodynamic therapy is increased by at least 40% compared to a composition which is devoid of a penetration enhancer and chelating agent. Preferably, the penetration of 5-carbon aminoketone in the skin of a patient in the photodynamic therapy is increased by at least about 50% compared to a composition which is devoid of a penetration enhancer and chelating agent. Preferably, the penetration of 5-carbon aminoketone in the skin of a patient in the photodynamic therapy is increased by at least about 60% compared to a composition which is devoid of a penetration enhancer and chelating agent. Preferably, the penetration of 5-carbon aminoketone in the skin of a patient in the photodynamic therapy is increased by at least about 70% compared to a composition which is devoid of a penetration enhancer and chelating agent. Preferably, the penetration of 5-carbon aminoketone in the skin of a patient in the photodynamic therapy is increased by at least about 80% compared to a composition which is devoid of a penetration enhancer and chelating agent.

Preferably, the penetration of 5-carbon aminoketone in the skin of a patient in the photodynamic therapy is increased by at least about 90% compared to a composition which is devoid of a penetration enhancer and chelating agent.

In yet another embodiment, the present invention provides a method for photodynamic therapy in the treatment of actinic keratosis, comprising administering a topical composition to the skin of a patient comprising:
a) a 5-carbon aminoketone compound of Formula I

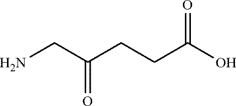

or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent.
wherein the method increases the penetration of 5-carbon aminoketone in the skin of a patient by at least about 30% when compared to the administration of a composition which is devoid of a penetration enhancer and chelating agent.

Preferably, the method increases the penetration of 5-carbon aminoketone in the skin of a patient by at least about 30%, at least about 40%, at least about 50%, at least about 60%, at least about 70%, at least about 80%, or at least about 90% compared to composition which is devoid of a penetration enhancer and chelating agent.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent.
wherein the method increases the penetration of 5-carbon aminoketone in the skin of the patient by at least about 30% when compared to the administration of a composition which is devoid of a penetration enhancer and chelating agent.

Preferably, the method increases the penetration of 5-carbon aminoketone in the skin of the patient by at least about 30%, at least about 40%, at least about 50%, at least about 60%, at least about 70%, at least about 80%, or at least about 90% compared to composition which is devoid of a penetration enhancer and chelating agent.

In yet another embodiment, the present invention provides a method for photodynamic therapy in the treatment of actinic keratosis, comprising administering a topical composition to the skin of a patient comprising:
a) a 5-carbon aminoketone compound of Formula I

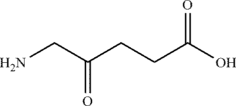

or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent.
wherein the method increases the penetration of 5-carbon aminoketone in the skin of the patient by at least about 30% when compared to the administration of a composition which is devoid of a penetration enhancer and chelating agent.

Preferably, the method increases the penetration of 5-carbon aminoketone in the skin of the patient to about at least about 30%, at least about 40%, at least about 50%, at least about 60%, at least about 70%, at least about 80%, or at least about 90% compared to a composition which is devoid of a penetration enhancer and chelating agent.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent.
wherein the method increases the penetration of 5-carbon aminoketone in the skin of the patient by at least about 30% when compared to the administration of a composition which is devoid of a penetration enhancer and chelating agent.

Preferably, the method increases the penetration of 5-carbon aminoketone in the skin of the patient by at least about 30%, at least about 40%, at least about 50%, at least about 60%, at least about 70%, at least about 80%, or at least about 90% compared to a composition which is devoid of a penetration enhancer and chelating agent.

In one embodiment, the method increases the penetration of 5-aminolevulinic acid in the skin of a patient by at least about 30% during photodynamic therapy of actinic keratosis comprising applying a pharmaceutical composition comprising 5-aminolevulinic acid on the affected area of the skin of a patient, wherein 5-aminolevulinic acid is present in an amount of about 20%w/w of the pharmaceutical composition.

In one embodiment, the method increases the penetration of 5-aminolevulinic acid in the skin of a patient by at least about 30% during photodynamic therapy of actinic keratosis comprising applying a pharmaceutical composition comprising 5-aminolevulinic acid on the affected area of the skin of a patient, wherein 5-aminolevulinic acid is present in an amount of 10%w/w of the pharmaceutical composition.

In yet another embodiment, the present invention provides a method for photodynamic therapy in the treatment of actinic keratosis, comprising administering a topical composition to the skin of a patient comprising:
a) a 5-carbon aminoketone compound of Formula I

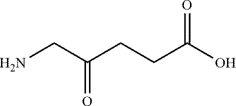

or its pharmaceutically acceptable salt in the form of a dry solid, and
b) a vehicle, wherein the vehicle comprises:
   (i) at least one penetration enhancer,
   (ii) at least one chelating agent, and
   (iii) optionally, an antifoaming agent,
wherein, the method increases the conversion of 5-carbon aminoketone compound to protoporphyrin IX production in the skin of the patient by at least about 30% when compared to the administration of a composition which is devoid of a penetration enhancer and chelating agent.

Preferably, the conversion of 5-carbon aminoketone compound to protoporphyrin IX production in the skin of a patient in the photodynamic therapy is increased by at least about 30% compared to a composition which is devoid of a penetration enhancer and chelating agent. Preferably, the conversion of 5-carbon aminoketone compound to protoporphyrin IX production in the skin of a patient in the photodynamic therapy is increased by at least 40% compared to a composition which is devoid of a penetration enhancer and chelating agent. Preferably, the conversion of 5-carbon aminoketone compound to protoporphyrin IX production in the skin of a patient in the photodynamic therapy is increased by at least 50% compared to a composition which is devoid of a penetration enhancer and chelating agent. Preferably, the conversion of 5-carbon aminoketone compound to protoporphyrin IX production in the skin of a patient in the photodynamic therapy is increased by at least 60% compared to a composition which is devoid of a penetration enhancer and chelating agent. Preferably, the conversion of 5-carbon aminoketone compound to protoporphyrin IX production in the skin of a patient in the photodynamic therapy is increased by at least 70% compared to a composition which is devoid of a penetration enhancer and chelating agent. Preferably, the conversion of 5-carbon aminoketone compound to protoporphyrin IX production in the skin of a patient in the photodynamic therapy is increased by at least 80% compared to a composition which is devoid of a penetration enhancer and chelating agent. Preferably, the conversion of 5-carbon aminoketone compound to protoporphyrin IX production in the skin of a patient in the photodynamic therapy is increased by at least 90% compared to a composition which is devoid of a penetration enhancer and chelating agent.

In yet another embodiment, the present invention provides a method for photodynamic therapy in the treatment of actinic keratosis, comprising administering a topical composition to the skin of a patient comprising:
a) a 5-carbon aminoketone compound of Formula I

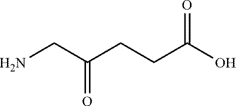

or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent,
wherein, the method increases the conversion of 5-carbon aminoketone compound to protoporphyrin IX production in the skin of the patient by at least about 30% when compared to the administration of a composition which is devoid of a penetration enhancer and chelating agent.

Preferably, the method increases the conversion of 5-carbon aminoketone compound to protoporphyrin IX production in the skin of a patient by at least about 30%, at least about 40%, at least about 50%, at least about 60%, at least about 70%, at least about 80%, or at least about 90% compared to a composition which is devoid of a penetration enhancer and chelating agent.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) at least one penetration enhancer selected from a group consisting of glycol derivatives, polyethylene glycol derivatives, and ethoxylated ether derivatives,
   (ii) ethylenediaminetetraacetic acid (EDTA) or its pharmaceutically acceptable salts thereof, and
   (iii) optionally, an antifoaming agent,
wherein, the method increases the conversion of 5-carbon aminoketone compound to protoporphyrin IX production in the skin of the patient by at least about 30% when compared to the administration of a composition which is devoid of a penetration enhancer and chelating agent.

Preferably, the method increases the conversion of 5-carbon aminoketone compound to protoporphyrin IX production in the skin of a patient by at least about 30%, at least about 40%, at least about 50%, at least about 60%, at least about 70%, at least about 80%, or at least about 90% compared to a composition which is devoid of a penetration enhancer and chelating agent.

In yet another embodiment, the present invention provides a method for photodynamic therapy in the treatment of actinic keratosis, comprising administering a topical composition to the skin of a patient comprising:
a) a 5-carbon aminoketone compound of Formula I

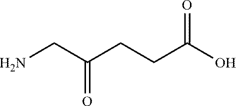

or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent,
wherein, the method increases the conversion of 5-carbon aminoketone compound to protoporphyrin IX production in the skin of the patient by at least about 30% when compared to the administration of a composition which is devoid of a penetration enhancer and chelating agent.

Preferably, the method increases the conversion of 5-carbon aminoketone compound to protoporphyrin IX production in the skin of a patient by at least about 30%, at least about 40%, at least about 50%, at least about 60%, at least about 70%, at least about 80%, or at least about 90% compared to a composition which is devoid of a penetration enhancer and chelating agent.

In yet another embodiment, the present invention provides a method for photodynamic therapy in the treatment of actinic keratosis, comprising administering a topical composition to the skin of a patient comprising:
a) 5-ALA or its pharmaceutically acceptable salt in the form of a dry solid, and
b) a vehicle, wherein the vehicle comprises:
   (i) at least one penetration enhancer selected from a group consisting of propylene glycol, polyethylene glycol, and 2-(2-Ethoxyethoxy)ethanol,
   (ii) ethylenediaminetetraacetic acid (EDTA) or its pharmaceutically acceptable salts thereof, and
   (iii) optionally, an antifoaming agent.
wherein, the method increases the conversion of 5-carbon aminoketone compound to protoporphyrin IX production in the skin of the patient by at least about 30% when compared to the administration of a composition which is devoid of a penetration enhancer and chelating agent.

Preferably, the method of the instant invention increases the conversion of 5-carbon aminoketone compound to protoporphyrin IX production in the skin of a patient by at least about 30%, at least about 40%, at least about 50%, at least about 60%, at least about 70%, at least about 80%, or at least about 90% compared to a composition which is devoid of a penetration enhancer and a chelating agent.

In one embodiment, the method increases the conversion of 5-aminolevulinic acid to protoporphyrin IX production in the skin of a patient by at least about 30% during photodynamic therapy of actinic keratosis comprising applying a pharmaceutical composition comprising 5-aminolevulinic acid on the affected area of the skin of a patient, wherein 5-aminolevulinic acid is present in an amount of about 20%w/w of the pharmaceutical composition.

In one embodiment, the method increases the conversion of 5-aminolevulinic acid to protoporphyrin IX production in the skin of a patient by at least 30% during photodynamic therapy of actinic keratosis comprising applying a pharmaceutical composition comprising 5-aminolevulinic acid on the affected area of the skin of a patient, wherein 5-aminolevulinic acid is present in an amount of 10%w/w of the pharmaceutical composition.

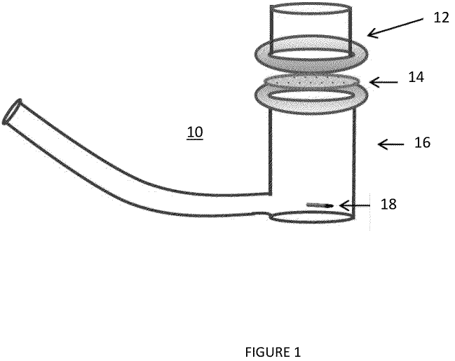

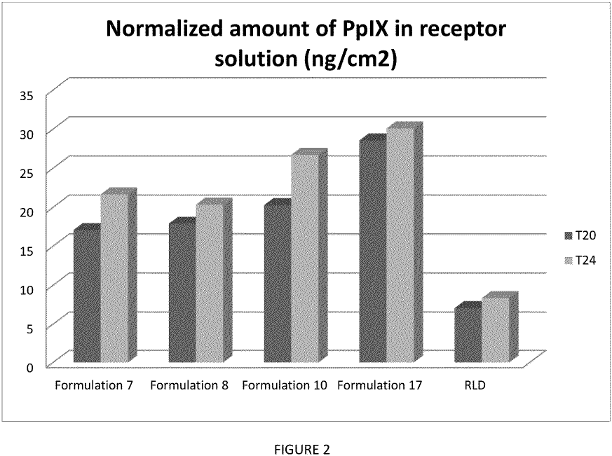

The amount of permeation of 5-aminolevulinic acid and conversion of 5-aminolevulinic acid to PpIX was determined in an in-vitro skin model shown in FIG. 1 for formulations comprising a permeation enhancer and a chelating agent. Formulations 6, 7, 8, 10, 17 and RLD were tested using female abdominal skin in an in-vitro skin model for permeation of 5-ALA by determining the amount of PpIX in a receptor solution and in the skin. The drug that permeates across the skin in an in vitro status dictates the amount available for subcutaneous or deeper skin tissues and systematic circulation. ALA is a naturally occurring amino acid that is ultimately converted into PpIX within the skin. As shown in FIG. 2, it was observed that Formulation 17, containing propylene glycol (“PG”) at an amount of 30% w/w with 4% Transcutol® and EDTA at 0.2% w/w showed the highest permeation with the highest quantities of PpIX present in the receptor phase at 20 hr and 24 hr followed by Formulation 10, containing propylene glycol at an amount of 40% w/w and EDTA at 0.25% w/w and Formulation 8, containing propylene glycol at an amount of 30% w/w and EDTA at 0.2% w/w. The formulations comprising PG and EDTA showed significantly higher permeation compare to RLD. Addition of 0.25% EDTA showed 2.5 to 2.9 times higher PpIX in the receptor solution compared with the RLD.

In one embodiment, the affected area includes upper extremities (e.g., the dorsal surface of the hands, upper arms or forearms), scalp or facial areas and other areas of the patient (e.g., the legs or portions of the arms other than the forearms).

The method includes applying the topical composition of the invention to the affected area and incubating with or without occlusion followed by illuminating the affected area with a light source. Preferably, the method includes covering the affected area with occlusion after application of the topical composition prior to illuminating the affected area with a light source. The occlusion may be a light-blocking occlusive dressing or a moisture protecting occlusion dressing. Preferably, the occlusion is a low density polyethylene barrier or a transparent film dressing.

In one embodiment, a method of enhancing penetration of a topical composition of 5-aminolevulinic acid (ALA) into tissue for photodynamic therapy is further provided. The method includes topically applying ALA to a treatment area to be treated with photodynamic therapy. The method further includes, after the ALA is applied to the treatment area, covering the treatment area with a polymeric barrier to have a degree of occlusion of 65% or more.

Preferably, the method includes after the ALA is applied to the treatment area, covering the treatment area with a low density polyethylene barrier. The treatment area is covered with the low density polyethylene barrier prior to light treatment to minimize transepidermal water loss from the treatment area. See U.S. Pat. No. 10,357,567, which is incorporated by reference in its entirety, for the techniques, methods, compositions, and devices related to PDT and PD.

In one embodiment, the light source used in the photodynamic therapy includes lasers, dye lasers pumped by argon or metal vapour lasers and frequency-doubled Nd:YAG lasers. Non-laser sources including tungsten filament, xenon arc, metal halide and fluorescent lamps halogen bulbs, LED light delivery and direct sunlight.

For effective treatment, it is desirable to have a power output that is uniform in intensity and color. Illuminators, such as those disclosed in U.S. Pat. Nos. 8,758,418; 8,216,289; 8,030,836; 7,723,910; 7,190,109; 6,709,446; and 6,223,071 and International Published Pa. Application No. WO 2017/066270, which are incorporated by reference in their entireties for the techniques, methods, compositions, and devices related to PDT and PD, are typically used to provide the proper uniformity of light for treatment purposes. These devices generally include a light source (e.g., a fluorescent tube, LED or LED array), coupling elements that direct, filter or otherwise conduct emitted light so that it arrives at its intended target in a usable form, and a control system that starts and stops the production of light when necessary. Preferably, the light source includes a plurality of LEDs, e.g., in an array.

In at least one embodiment, when ALA is used for the treatment of actinic keratosis, the LED preferably emits blue light having wavelengths at or above 400 nanometers (nm), for example, about 430 nm, about 420 nm or, for example, 417 nm. However, the LED may also emit visible light in other ranges of the spectrum, such as in the green and/or red ranges between 400 and 700 nm, for example, about 625 nm to 640 nm or, for example, 635 nm. For example, the LED may also emit light having wavelengths of 510 nm, 540 nm, 575 nm, 630 nm, or 635 nm. In addition, the LED may be configured to emit light continuously or the LED may be configured to flash the diodes on and off based on a predetermined interval. Furthermore, the LED may be configured such that only one wavelength of light (e.g., blue) is emitted. Alternatively, the LED may be configured such that two or more wavelengths of light are emitted from the arrays. For example, the LED may be configured to alternately emit blue light and red light for treatment purposes. In one embodiment, the LED arrays may also emit red light having wavelengths of 570 to 670 nm.

In one embodiment of the present disclosure, blue light having a wavelength of approximately 417 nm is applied at an intensity of 10 mW/cm² for 1000 seconds to provide a dose of 10 J/cm². However, the intensity may be increased (for example, doubled) to provide 20 J/cm², to reduce the treatment time. For example, the intensity may be increased so as to reduce the treatment time by about one-half. Preferably, the blue light is applied for a period in the range of 5 to 15 minutes. In other embodiments, red light (such as red light generated by light emitting diodes (LEDs) at, for example, 635 nm) may be used. The red light can provide a dose of, for example, 10 to 75 J/cm² (such as 37 J/cm²), e.g., within 10 minutes.

In another embodiment, the illuminator may irradiate the lesions with a uniform intensity red light for a prescribed period. In certain embodiments, the illuminator irradiates the lesions with a uniform intensity blue light for a first prescribed period and then irradiates the lesions with a uniform intensity red light for a second prescribed period. For example, in some embodiments, the illuminator is configured to irradiate the lesions with a uniform intensity blue light (e.g., 417 nm) at a low intensity (e.g., about 0.1 J/cm² to about 2 J/cm²) to photobleach, for example, protoporphyrin IX (PpIX) present at the surface of the patient’s skin, and irradiate the lesions with a uniform intensity red light (e.g., 635 nm) at a high intensity (e.g., about 30 J/cm² to about 150 J/cm²) to activate PpIX present at deeper layers of the patient’s skin, thus avoiding potential damage to the upper layers of the patient’s skin.

Furthermore, since the total light dose (J/cm²) is equal to irradiance (W/cm²) multiplied by time (sec), an additional parameter to be controlled for delivery of the correct treatment light dose is exposure time. This may be accomplished by a timer, which can control the electrical power supplied to LED arrays appropriately, and which can be set by the physician. Data has shown that 10 J/cm² delivered from a source with an irradiance density of 10 mW/cm², or an irradiance density of about 9.3 to about 10.7 mW/cm², produces clinically acceptable results for desired treatment areas (e.g., face, scalp, extremities). An adjustable illuminator may deliver an irradiance density of 20 mW/cm² for an exposure time of 500 seconds (8 min. 20 sec) to deliver a clinically acceptable light dose of 10 J/cm². In certain embodiments, a lower intensity may be used with a longer exposure time (e.g., 1,000 seconds of exposure time for a light dose of 10 J/cm²). Alternatively, the illuminator may include higher power ranges, such as 30 mW/cm², over an exposure time resulting in a light dose of 10 J/cm². A selected light dose may also be administered by additionally or alternatively varying the irradiance density over treatment time.

Following a first treatment period, light may be applied for a second treatment period, e.g., about 8-15 minutes. In at least one embodiment, a heat source may be used and may be an infrared quartz heater. In at least one embodiment, the heat source may comprise frame mounted resistance tape heaters or a plurality of heaters, including at least one selected from the group including IR LEDs, resistance cartridge heaters, positive temperature coefficient heaters, or IR quartz heaters, as mentioned above. The heat may be deliberately generated and directed towards the area to be treated, as opposed to ambient heat in the clinical setting or by-product heat from one or more operating mechanisms of the illuminator.

The method further includes heating the affected area where the lesion is present before or during illumination of the area. In at least one embodiment, a heating element (a heat source) may be provided. The heat source may be used to heat the region to be treated.

According to one embodiment, a method of treatment includes warming up an illuminator so as to cause heat to be emitted from the illuminator, and exposing a treatment site to the illuminator. The heat accelerates the conversion of the ALA to porphyrin (e.g., photosensitive porphyrin or proto porphyrin). The relationship between temperature exposure and ALA conversion is non-linear, and the enzymatic pathways responsible for the conversion are highly sensitive to temperature. In at least one embodiment, increasing the temperature of tissue by approximately 2° C. may approximately double the rate of production of protoporphyrin IX (PpIX), for example. The heat may be applied before or during illumination with the illuminator.

For example, first, the ALA may be applied. Next, the heating element may be activated, to apply heat to the patient’s skin for a first treatment period for a thermal soak, which may be 20-30 minutes, for example. During the heating, the treatment site may or may not be occluded. In other words, the treatment site may be heated while being occluded.

Following the first treatment period, light may be applied for a second treatment period, e.g., about 8-15 minutes. In at least one embodiment, the heat source may be an infrared quartz heater. In at least one embodiment, the heat source may comprise frame mounted resistance tape heaters or a plurality of heaters, including at least one selected from the group including IR LEDs, resistance cartridge heaters, positive temperature coefficient heaters, or IR quartz heaters, as mentioned above. The heat may be deliberately generated and directed towards the area to be treated, as opposed to ambient heat in the clinical setting or by-product heat from one or more operating mechanisms of the illuminator.

In some embodiments, the illuminator may be provided with a fan or other air distributor which provides a gentle flow of air (e.g., a laminar or other generally even flow) tangential to the skin surface, which may reduce the sensation of pain.

The topical composition is prepared by mixing 5-aminolevulinic acid and a vehicle to form the composition ready to be applied to the skin of the patient for photodynamic therapy in the treatment of dermatological diseases, including actinic keratosis, disseminated superficial actinic porokeratosis (DSAP) or refractory disseminated porokeratosis, acne (e.g., cystic acne, inflammatory acne, non-inflammatory acne). The dermatological diseases are not limited to the foregoing. The dermatological diseases also encompass skin abnormalities such as contact dermatitis, rash, housewives’ eczema, atopic dermatitis, seborrheic dermatitis, lichen Vidal, prurigo, warts, drug eruption, photo-damaged skin, solar dermatitis, pruritus cutaneous, psoriasis, acne vulgaris, erythema, as well as cell proliferation disorders including cancers (e.g., non-melanoma cancer including keratinocyte carcinoma or other malignant skin cancers). The dermatological diseases include, for example, basal cell carcinoma (including infiltrative basal cell carcinoma, nodular basal cell carcinoma, recurrent nodular basal cell carcinoma, multi-focal basal cell carcinoma), squamous cell carcinoma, Bowen’s disease, solar keratosis, and subcutaneous cancer.

One example of a treatment method for precancerous lesions, such as actinic keratosis, by photodynamic therapy with ALA is now described. Essentially anhydrous ALA is admixed with the vehicle just prior to its use. The anhydrous ALA may be, for example, the hydrochloride salt of aminolevulinic acid (ALA), an endogenous 5-carbon aminoketone. In at least one embodiment, the ALA is contained in powderized form inside a first ampule. A second ampule contains a solution vehicle. The first and second ampules are contained inside a plastic applicator. The first and second ampules may be crushed, e.g., by applying finger pressure, or inside a device configured to exert pressure on the ampules. Once the ampules are crushed, the ALA formerly contained in the first ampule contacts the solution formerly contained in the second ampule, and dissolves in the solution vehicle. The applicator in which the ampules were provided may be shaken so as to disperse and dissolve the powdered ALA in the solution vehicle. Once combined, the resulting solution is applied to the patient within 2 hours of preparation.

In some embodiments, the ALA may be provided in a composition such as a ready-to-use solution or a reconstituted powder for solution, gel, cream or lotion formulation. In another embodiment, the composition comprises 5-aminolevulinic acid hydrochloride in an amount of about 10% to about 70% w/w based on the total weight of the composition, preferably from about 20% to about 50% w/w based on the total weight of the composition. In one embodiment, ALA may be applied in a topical composition with a concentration of 20%. The ALA admixture is topically applied to the lesions using a point applicator to control dispersion of the ALA admixture, in at least one embodiment, so as to achieve a substantially uniform wetting of the lesion surface with the ALA by contacting the ALA with the lesion surface. The term “substantial,” or “substantially” as used herein, may refer to any value which lies within the range as defined by a variation of up to ±15% from the average value. However, in other embodiments, the ALA may be applied digitally (i.e., by first disposing the ALA on the gloved fingertips of a practitioner, who then dabs the ALA on the region to be treated), or with a tool such as a spatula. Preferably, a treatment method for actinic keratosis of upper extremities further includes covering the lesions with occlusion after application of the topical composition prior to illuminating with a light source. The occlusion may be a light-blocking occlusive dressing or a moisture protecting occlusion dressing. Preferably, the occlusion is a low density polyethylene barrier or a transparent film dressing. The occlusion may be held in place with an elastic net dressing.

In certain applications, materials other than low density polyethylene may be used as long as they have a degree of occlusion of 65% or more. In certain applications, certain materials may be used as long as they have a degree of occlusion of 75% or more.

### EXAMPLES

The following examples more specifically illustrate compositions and methods according to various embodiments described herein. These examples should in no way be construed as limiting the scope of the present technology.

### Example 1 Preparation of Topical Compositions

The Table below provides the composition of representative Formulations 1 to 19 according to embodiments of the invention.

TABLE 1

Topical compositions

| | | | | | | | | | | | | | | | | | | | | |
|---|---|---|---|---|---|---|---|---|---|---|---|---|---|---|---|---|---|---|---|---|
| Ingredients | RLD* | 1 | 2 | 3 | 4 | 5 | 6 | 7 | 8 | 9 | 10 | 11 | 12 | 13 | 14 | 15 | 16 | 17 | 18 | 19 |
| Alcohol, USP | 44.37 | 24.37 | 43.46 | 44.37 | 24.37 | 24.37 | 14.37 | 24.37 | 14.42 | 24.37 | 4.37 | 24.37 | 24.37 | 24.37 | 24.37 | 24.37 | 10.37 | 10.42 | 4.42 | 24.37 |
| Purified Water | 43.36 | 43.46 | 24.37 | 43.36 | 43.36 | 43.36 | 43.21 | 43.21 | 43.21 | 43.21 | 43.21 | 43.06 | 42.96 | 42.71 | 43.36 | 43.46 | 43.21 | 43.21 | 43.21 | 43.21 |
| Laureth-4 | 6.59 | 6.59 | 6.59 | 6.59 | 6.59 | 6.59 | 6.59 | 6.59 | 6.59 | 6.59 | 6.59 | 6.59 | 6.59 | 6.59 | 6.59 | 6.59 | 6.59 | 6.59 | 6.59 | 3.3 |
| Polyethylene Glycol 400 | 1.65 | 1.65 | 1.65 | 1.65 | 1.65 | 1.65 | 1.65 | 1.65 | 1.65 | 1.65 | 1.65 | 1.65 | 1.65 | 1.65 | 1.65 | 1.65 | 1.65 | 1.65 | 1.65 | 1.65 |
| Isopropyl Alcohol 99%, USP | 3.93 | 3.93 | 3.93 | 3.93 | 3.93 | 3.93 | 3.93 | 3.93 | 3.93 | 3.93 | 3.93 | 3.93 | 3.93 | 3.93 | 3.93 | 3.93 | 3.93 | 3.93 | 3.93 | 3.93 |
| Propylene Glycol | 0 | 20 | 20 | 0 | 30 | 20 | 30 | 20 | 30 | 20 | 40 | 20 | 20 | 20 | 20 | | 40 | 30 | 30 | 20 |
| Diethylene Glycol Monoethyl Ether (Transcutol®) | 0 | 0 | 0 | 0 | 4 | 0 | 0 | 0 | 0 | 0 | 0 | 0 | 0 | 0 | 0 | 20 | 4 | 4 | 10 | 0 |
| Edetate Calcium Disodium | 0 | 0 | 0 | 0 | 0 | 0 | 0 | 0 | 0 | 0 | 0 | 0 | 0 | 0 | 0.1 | 0 | 0 | 0 | 0 | 0 |
| Disodium Edetate (EDTA) | 0 | 0 | 0 | 0.1 | 0.2 | 0.1 | 0.25 | 0.25 | 0.2 | 0.25 | 0.25 | 0.4 | 0.5 | 0.75 | | | 0.25 | 0.2 | 0.2 | 0.25 |
| Cyclomethicone | | | | | 0.5 | | | | | | | | | | | | | | | |
| ALA HCl | 20 | 20 | 20 | 20 | 20 | 20 | 20 | 20 | 20 | 20 | 20 | 20 | 20 | 20 | 20 | 20 | 20 | 20 | 20 | 20 |

*RLD herein refers to compositions devoid of a penetration enhancer and chelating agent.

### Method of Preparation

A composition, containing ALA, is prepared by admixing ALA with the vehicle. The vehicle was first prepared by mixing the ingredients in the following order: To the purified water, chelating agent such as edetate disodium or edetate calcium disodium was added and mixed well. Then ethyl alcohol was added, then propylene glycol was added, then polyethylene glycol 400, then isopropyl alcohol, then Transcutol®,and then Laureth-4 was added. The vehicle was mixed after addition of each ingredient. Finally, cyclomethicone was added to get the final mix. For compositions without a chelating agent, the vehicle was prepared by mixing the ingredients in any order.

### Example 2 Permeation of 5-ALA Determined

Formulations 6, 7, 8, 10, 17 and RLD were tested using a special in-vitro skin permeation testing model (IVPT) 10 shown in FIG. 1, which is designed to determine the amount of PpIX in the receptor solution, amount deposited in the skin, and the amount remaining on the skin surface.

The IVPT set up used in this investigation comprised of static Franz cells (2.5 ml) with a donor compartment 12, a membrane 14, and a receptor compartment 16 as shown in FIG. 1. Full thickness human skin was used in these studies. The skin temperature was maintained at 40±1° C. through immersion into a temperature controlled water bath (44° C.), a stirring plate (such as magnetic stirrer 18 shown in FIG. 1) was used to ensure a homogenised receptor solution of 30 mM CPC in phosphate buffer saline (PBS). 5-ALA and placebo formulation was supplied separately and mixed just before dose application. A dose of 30 mg±10% was applied manually for each cell; a 0.5 ml of the receptor solution was sampled at t0 prior to application to ensure no cell contamination, followed by two samples, one at 20 hours (t20) and one at 24 hours (t24). The time point selection meant to maximize the detection of the PpIX in the receptor and to study the impact of blue light illumination (applied at 20 h, 20 mW, for 16.25 min) on the conversion of PpIX. At the end of the study, the skin surface in each cell was washed with three liquid washes (20% methanol in water) and one dry wipe. The remaining skin layer was then cut into small pieces and PpIX was extracted using a strong solvent, for example, dimethyl sulfoxide (DMSO).

All collected samples were quantified for PpIX in duplicate using fluorescence endpoint detection within a Synergy HTX Microplate reader.

As shown in FIG. 2, it was observed that Formulation 17 (Containing 30% PG, 4% TC and 0.2% EDTA) showed the highest permeation with highest quantities of PpIX present in the receptor phase at 20 hr and 24 hr followed by Formulation 10 (containing 40% PG and 0.25% EDTA) and Formulation 8 (containing 30% PG and 0.2% EDTA) and then Formulation 7 (containing 20% PG and 0.25% EDTA). The Formulations comprising PG and EDTA showed significantly higher permeation compare to RLD. The addition of 0.25% EDTA showed 2.5 to 2.9 times higher PpIX in the receptor solution compared with the RLD.

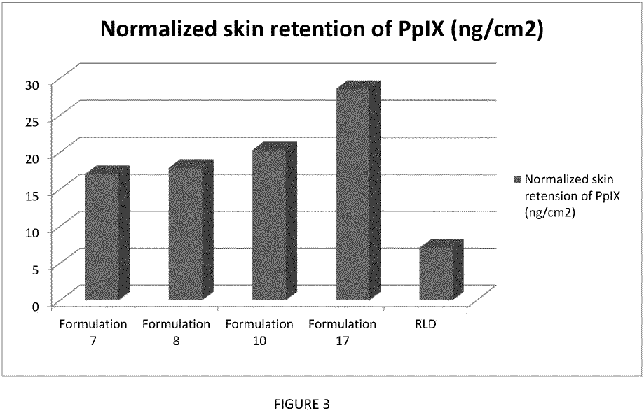

Skin retention values of PpIX also showed the highest retention for Formulation 17 (4.76 ng/cm²) and Formulation 10 (3.93 ng/cm²) as seen in FIG. 3.

### Effect of Change in Chelating Agent

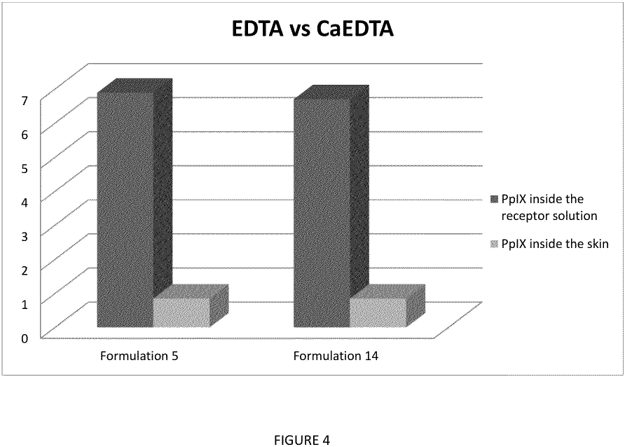

Formulation 5 (20% PG + 0.1% EDTA) and Formulation 14 (20% PG + 0.1% Edetate Calcium disodium) were tested for the amount of PpIX in receptor solution and in the skin. As shown in FIG. 4, there was no significant difference seen between the formulations.

### Example 3 Effect of Temperature on PpIX Formation

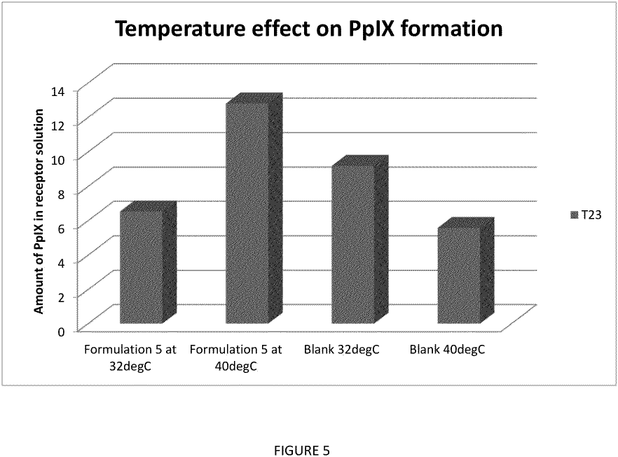

In this study, the impact of skin and cell environment temperature on PpIX content was assessed using Formulation 5 (20% PG + 0.1% EDTA). The study was repeated at two different temperatures using the same skin donor. The skin temperature for the first study was 32° C. and 40° C. for the second. Beside the temperature, all other study conditions were identical to the previously described IVPT method. The results of the study may be seen in FIG. 5. At 32° C., there is no significant difference between the applied sample and blank skin with regards to PpIX production. At 40° C., there is a significant difference between the applied sample and blank skin with regards to PpIX production. There is also a significant difference between the applied Formulation 4 at 32° C. and 40° C. when looking at the PpIX formation. In view of the foregoing, it is believed that temperature may have a significant impact on the formation of PpIX.

Additional advantages and modifications will readily occur to those skilled in the art. Therefore, the invention in its broader aspects is not limited to the specific details and representative compositions and methods, shown and described herein. Accordingly, various modifications may be made without departing from the spirit and scope of the general inventive concept as defined by the appended claims and their equivalents.

### Example 4 In-Vitro Skin Permeating Testing (IVPT) Study of PpIX

### 1. Preparation of Phosphate Buffered Solution (PBS)

Ten PBS tablets were taken in a one litre volumetric flask and 800 mL deionized water was added to it. The tablets were dissolved in water by placing a PTFE-coated magnetic stirrer bar. The bar was then removed and the volume was made to one liter using deionized water.

### 2. Preparation of PBS-CPC (Cetylpyridinium Chloride)

In order to prepare a 30 mM solution of PBS-CPC, approximately 5.37 g of CPC (Sigma-Aldrich) was weighed and transferred to a 500 mL volumetric flask. PBS was added to dissolve the CPC by sonication for 20 min (approx.) and made to the final volume.

### 3. Preparation of 20% (W/V) 5-Aminolevulinic Acid (5-ALA) Formulation

Approximately 70 mg of 5-ALA was weighed and taken in to a micro centrifuge tube. 350 µL of a placebo formulation was added and the tube was vortexed for 30 sec to dissolve the drug in to the placebo formulation.

### 4. Preparation of 20% Methanol Solution

In a reagent bottle, 100 mL of methanol and 400 mL of deionized water were added. The solution was mixed using a PTFE-coated magnetic stirrer bar.

### 5. Permeation Study

The permeation study was conducted using full thickness human epidermis (female abdominal). The permeation study of 5-ALA formulations was conducted using static vertical Franz diffusion cells. The average surface area of the cells was 0.6 cm² and the receptor compartment was filled up with 2.25 mL of PBS-CPC (30 mM). Skin was cut using a cork borer and rinsed gently in deionized water in order to remove excess tissue residue. A piece of filter paper was placed underneath the skin (dermal side) and in between donor and receptor. The two compartments were tightly held together by using a metallic screw clap. The Franz cell set up, skin integrity test and dose application were conducted:
Skin integrity was checked using a Trans Epidermal Electrical Resistance (TEER) reader using PBS on both cell compartments.
Cells with skin TEER value ≤20 KΩ were excluded.

PBS from both compartments was removed and the receptor compartment was filled with 2.25 mL of PBS-CPC and the donor compartment was dried using tissue paper. The cells were placed in a water bath where the temperature was set at 43.5 to 45° C. to maintain skin surface temperature of 40±1° C. A stirring rate of 350 rpm was applied. Skin temperature was monitored at regular intervals using a blank skin. Once the skin temperature reached the target temperature, 30 mg of freshly prepared 20% 5-ALA formulation was applied on the epidermal side of the skin. 500 µL samples from the receptor compartment were taken at 0, 20 and 24 hours. Each time, it was replaced by the same volume of fresh temperature equilibrated PBS. The total duration of the permeation study was 24 hours. At 20 hr, after collecting the samples, blue light (20 mW) was applied on the cells for 16 min and 25 sec. The number of replicates (n) for each formulation was 4 to 5. A blank cell was also used as a control to determine any possible interference. All the permeation samples were analyzed using a Synergy HTX microplate reader. In a 96 well plate, 200 µL permeation samples were placed in duplicate and analyzed.

### 6. Wash and Skin Extraction

After 24 h, the process was conducted as below:
The skin surface was washed three times with a 20% methanol solution and then gently wiped on the surface with a cotton swab. The samples were taken up in a Scintillation vial and vortexed for 3 sec.
After washing, the skin was cut into pieces and taken in to an Eppendorf tube with 1 mL of DMSO. The tube was sonicated for 20 min followed by centrifugation for 10 min at 15000 rpm and at 21° C. The supernatant solution was analyzed.

100 µL of washing and skin samples were placed in a 96 well plate in duplicate along with 100 µL of blank PBS-CPC (30 mM) and analyzed using Synergy HTX microplate reader.

### 7. Effect of EDTA Level on PpIX at 0.1%, 0.15% and 0.2%

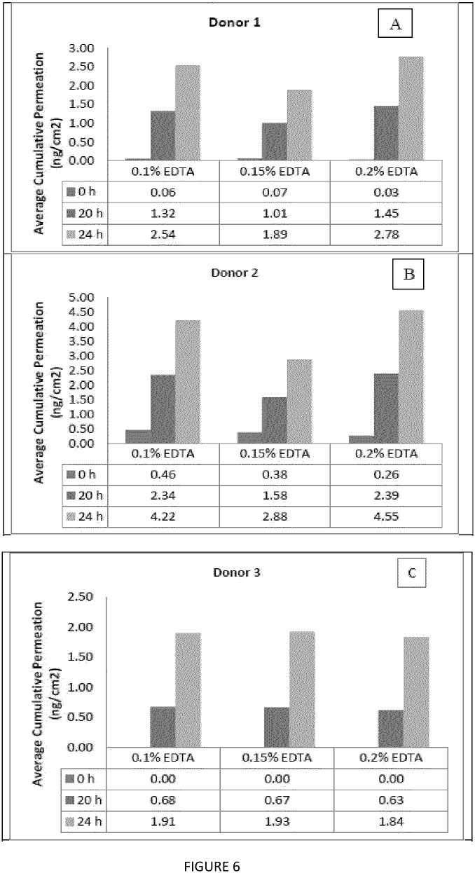

The effect of PpIX production from three formulations finished products with EDTA concentrations 0.1%, 0.15% and 0.2%, respectively, were prepared and were compared on three different donors. Summary of the PpIX amount quantified in the receptor solution at t₀, t₂₀ and t₂₄ are presented in FIG. 6.

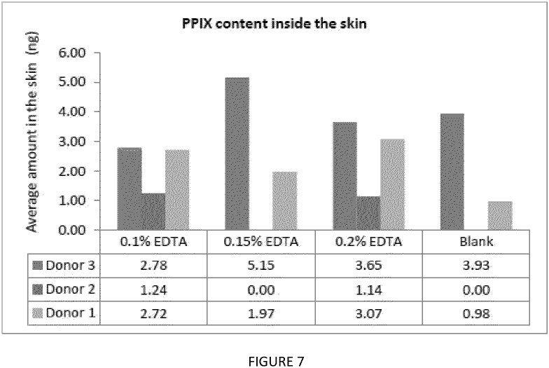

In addition, FIG. 7 shows the amount of PpIX created in the skin compartment of the three donors tested wherein the data is shown for the content quantified inside the skin compartment for donors 1, 2, and 3.

Based on the findings of the experiments, the analysis showed no statistical difference between the three levels of EDTA concentration. This supports that the use of a lower EDTA levels (<0.2%) could result in equivalent PpIX levels compared with 0.2% EDTA.

### Example 5 Effect of Varying the Ingredients in the Formulation

Studies were conducted to determine synergism between the components of the compositions of the present disclosure. Ingredients of the instant formulations, viz., Transcutol®,propylene glycol (PG) and EDTA, were investigated using 5 different formulation strategies. Details of the formulation are provided herein below Tables 2a and 2b.

TABLE 2A

List of formulations tested

| Ingredients | D | E | X | B | Y |
|---|---|---|---|---|---|
| | (w/w%) | | | | |
| Water | 46.96 | 47.16 | 46.96 | 42.96 | 46.96 |
| EDTA | 0.2 | 0 | 0.2 | 0.2 | 0 |
| Propylene Glycol (PG) | 30 | 30 | 0 | 30 | 0 |
| Polyethylene Glycol 400 | 1.65 | 1.65 | 1.65 | 1.65 | 1.65 |
| Isopropyl Alcohol | 3.93 | 3.93 | 3.93 | 3.93 | 3.93 |
| Transcutol® | 0 | 0 | 0 | 4 | 4 |
| Alcohol (Ethanol), USP | 10.42* | 10.42* | 40.42* | 10.42* | 36.62* |
| Laureth-4 | 6.59 | 6.59 | 6.59 | 6.59 | 6.59 |
| Cyclomethicone | 0.25 | 0.25 | 0.25 | 0.25 | 0.25 |
| ALA HCL | 20 | 20 | 20 | 20 | 20 |

TABLE 2B

Additional formulations are as follows

| Ingredients | F | P | Q | R | S |
|---|---|---|---|---|---|
| | (w/w%) | | | | |
| Water | 45.46 | 48.15 | 49.15 | 41.35 | 48.48 |
| EDTA | 0.3 | 0.1 | 0.15 | 0.1 | 0.15 |
| Propylene Glycol (PG) | 25 | 10 | 21 | 33 | 37.50 |
| Polyethylene Glycol 400 | 2.15 | 2.95 | 3.25 | 3.30 | 1.94 |
| Isopropyl Alcohol | 4.75 | 2.96 | 4.63 | 2.45 | 3.20 |
| Transcutol® | 3.5 | 3 | 1.5 | 0 | 0 |
| Alcohol (Ethanol), USP | 11.0* | 26.64* | 13.25* | 13.05* | 3* |
| Laureth-4 | 7.49 | 5.85 | 6.92 | 6.25 | 5.35 |
| Cyclomethicone | 0.35 | 0.35 | 0.15 | 0.5 | 0.38 |
| ALA HCL | 20 | 20 | 20 | 20 | 20 |

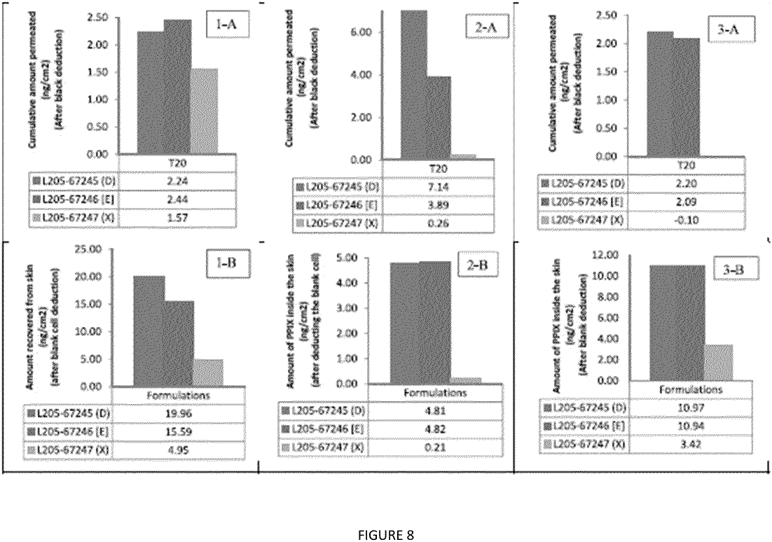

The synergism study for the formulations was performed on three donors. PpIX levels were quantified in the receptor solution (Permeated) and inside the skin. Comparing the results from formulations D, E and X demonstrated the synergetic effect between EDTA and PG. PpIX levels in both the receptor compartment (Permeated) and inside the skin were significantly higher from formulation D when compared with formulation X containing EDTA only (FIG. 8).

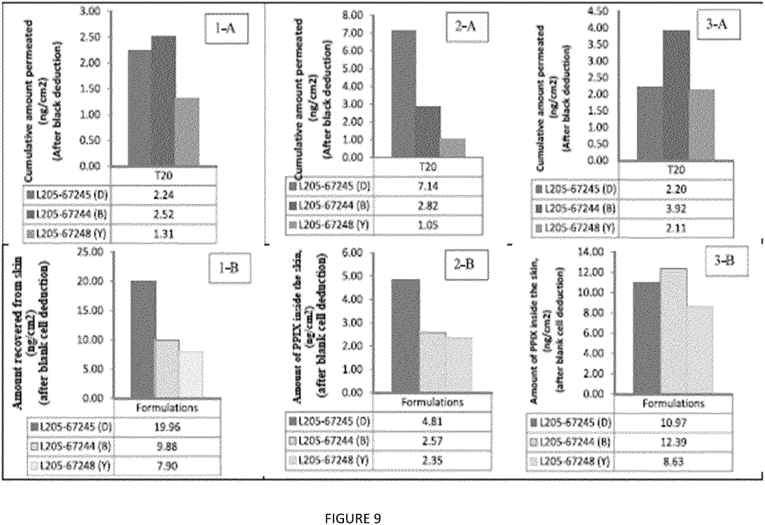

In the second experiment, a synergetic effect between Transcutol®,PG and EDTA was investigated (FIG. 9). PpIX levels quantified following the application of formulation B containing Transcutol®,PG and EDTA were significantly higher when compared to formulation Y containing Transcutol® only. Comparison of PpIX levels from formulation B and D did not show superiority of formulation B. PG and EDTA may be the main drivers for the PpIX enhanced levels, whereas Transcutol® may slow the release of the ALA via increased retention on the skin surface.

Although the figures and description may illustrate a specific order of method steps, the order of such steps may differ from what is depicted and described, unless specified differently above. Also, two or more steps may be performed concurrently or with partial concurrence, unless specified differently above.

1. A method for the treatment of a dermatological disorder, comprising:
applying, to an affected area of the skin of a patient, a topical composition comprising:
a) a 5-carbon aminoketone compound of Formula I 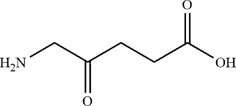
or a pharmaceutically acceptable salt thereof; and
b) a vehicle,
wherein the vehicle comprises:
(i) at least one penetration enhancer, and
(ii) at least one chelating agent, and
wherein the topical composition is applied to the skin for a period of about 15 minutes to about 10 hours, followed by illuminating the affected area of the skin with a light source.

2. The method of claim 1, wherein the vehicle further comprises an antifoaming agent.

3. The method of claim 1,wherein the method of treatment comprises photodynamic therapy.

4. The method of claim 1,wherein the compound of Formula I or a salt thereof is in the form of a dry solid.

5. The method of claim 1,further comprising heating the affected area of the skin before or during illumination of the area.

6. The method of claim 1,further comprising covering the affected area of the skin with occlusion after application of the topical composition and prior to illuminating the affected area with the light source.

7. The method of claim 6, wherein the occlusion is a light-blocking occlusive dressing.

8. The method of claim 6, wherein the occlusion is a transparent film dressing.

9. The method of claim 6, wherein the occlusion is a low density polyethylene barrier.

10. The method of claim 1,wherein the light source delivers blue light.

11. The method of claim 1,wherein the light source delivers red light.

12. The method of claim 1,wherein the light source delivers sunlight.

13. The method of claim 10, wherein blue light is delivered at 10 to 20 J/cm².

14. The method of claim 11, wherein red light is delivered at 10 to 75 J/cm².

15. The method of claim 1,wherein the compound of Formula I or a salt thereof is aminolevulinic acid hydrochloride.

16. The method of claim 1,wherein the at least one penetration enhancer is selected from a group consisting of dialkyl derivatives of acetamide and formamide, pyrrolidone derivatives, fatty acids, glycol derivatives, glycerides, azones, polysorbates, macrogolglycerides, polyethylene glycol derivatives, ethoxylated ether derivatives, bile salts, and sulfated glycosaminoglycan, or a combination of any two or more thereof.

17. The method of claim 16, wherein the at least one penetration enhancer is selected from a group consisting of glycol derivatives, polyethylene glycol derivatives, and ethoxylated ether derivatives, or a combination of any two or more thereof.

18. The method of claim 1, wherein:
(i) the at least one penetration enhancer is selected from a group consisting of propylene glycol, polyethylene glycol, and 2-(2-Ethoxyethoxy)ethanol; and
(ii) the at least one chelating agent is selected from ethylenediaminetetraacetic acid (EDTA) or a pharmaceutically acceptable salt thereof.

19. The method of claim 1,wherein the dermatological disorder is actinic keratosis.

20. The method of claim 18, wherein the dermatological disorder is actinic keratosis, and wherein the affected area of the skin includes the upper extremities.

21. The method of claim 18, wherein the dermatological disorder is actinic keratosis, and wherein the affected area of the skin includes a scalp or facial area.

22. The method of claim 20, further comprising, covering the affected area with occlusion after application of the topical composition and prior to illuminating the affected area of the skin with the light source.

23. The method of claim 22, wherein the occlusion is a light-blocking occlusive dressing.

24. The method of claim 22, wherein the occlusion is a transparent film dressing.

25. The method of claim 22, wherein the occlusion is a low density polyethylene barrier.

26. A method for photodynamic therapy in the treatment of a dermatological disorder, comprising administering to the skin of a patient a topical composition comprising:
a) a 5-carbon aminoketone compound of Formula I or a pharmaceutically acceptable salt thereof; 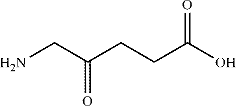
b) propylene glycol;
c) 2-(2-Ethoxyethoxy)ethanol; and
d) disodium edetate.

27. The method of claim 26, wherein the compound of Formula I is aminolevulinic acid hydrochloride.

28. The method of claim 26,wherein the compound of Formula I is present in an amount of about 1% w/w to about 30 % w/w of the composition.

29. The method of claim 28, wherein aminolevulinic acid hydrochloride is present in an amount of about 20% w/w of the composition.

30. The method of claim 26,wherein the propylene glycol is present in an amount of about 10% w/w to about 50% w/w of the composition.

31. The method of claim 26,wherein the 2-(2-Ethoxyethoxy)ethanol is present in an amount of about 2% w/w to about 50% w/w of the composition.

32. The method of claim 26,wherein the disodium edetate is present in an amount of about 0.1 about to 0.25% w/w of the composition.

33. The method of claim 26, wherein the composition further comprises an antifoaming agent.

34. The method of claim 33, wherein the antifoaming agent is cyclomethicone.

35. A topical composition for the treatment of a dermatological disorder, comprising:
a) a 5-carbon aminoketone compound of Formula -I 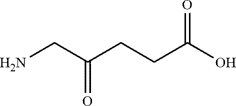
or its pharmaceutically acceptable salt, and
b) a vehicle,
wherein the vehicle comprises:
(i) at least one penetration enhancer, and
(ii) at least one chelating agent,
wherein the topical composition is applied for a period of about 15 minutes to about 10 hours.

36. A topical composition for the treatment of a dermatological disorder, comprising:
a) a 5-carbon aminoketone compound of Formula -I 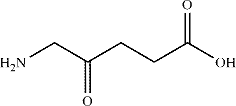
or its pharmaceutically acceptable salt, and
b) a vehicle,
wherein the vehicle comprises:
(i) at least one penetration enhancer, and
(ii) at least one chelating agent,
wherein the topical composition increases the conversion of 5-carbon aminoketone compound to protoporphyrin IX production in the skin of a patient by at least about 30%, compared to a composition which is devoid of a penetration enhancer and a chelating agent.

37. The composition of claim 35,wherein the vehicle further comprises an antifoaming agent.

38. The composition of claim 35,wherein the composition is utilized for photodynamic therapy.

39. The composition of claim 35,wherein after application of the composition, the affected area is illuminated with a light source.

40. The composition of claim 35,wherein the compound of Formula I is in the form of a dry solid.

41. The topical composition of claim 39, wherein the light source delivers blue light.

42. The topical composition of claim 39, wherein the light source delivers red light.

43. The topical composition of claim 39, wherein the light source delivers sunlight.

44. The topical composition of claim 41, wherein blue light is delivered at 10 to 20 J/cm².

45. The topical composition of claim 42, wherein red light is delivered at 10 to 75 J/cm².

46. The topical composition of claim 35, wherein the compound of Formula I is aminolevulinic acid hydrochloride.

47. The topical composition of claim 35, wherein:
(i) the at least one penetration enhancer is selected from a group consisting of propylene glycol, polyethylene glycol, and 2-(2-Ethoxyethoxy)ethanol, and
(ii) the at least one chelating agent is selected from ethylenediaminetetraacetic acid (EDTA) or a pharmaceutically acceptable salt thereof.

48. The topical composition of claim 35,wherein the dermatological disorder is actinic keratosis.

49. A topical composition for use in a photodynamic therapy for the treatment of a dermatological disorder, comprising:
a) a 5-carbon aminoketone compound of Formula I or a pharmaceutically acceptable salt thereof, 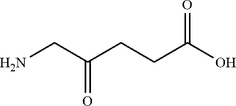
b) propylene glycol;
c) 2-(2-Ethoxyethoxy)ethanol; and
d) disodium edetate.

50. The topical composition of claim 49, wherein the compound of Formula I is present in an amount of about 1% w/w to about 30% w/w of the composition.

51. The topical composition of claim 50, wherein the compound of Formula I is present in an amount of about 20% w/w of the composition.

52. The topical composition of claim 49, wherein the propylene glycol is present in an amount of about 10% w/w to about 50% w/w of the composition.

53. The topical composition of claim 49, wherein the 2-(2-Ethoxyethoxy)ethanol is present in an amount of about 2% w/w to about 50 % w/w of the composition.

54. The topical composition of claim 49, wherein disodium edetate is present in an amount of about 0.1% to 0.25% w/w of the composition.

55. The topical composition of claim 49, wherein the composition further comprises an antifoaming agent.

56. The topical composition of claim 55, wherein the antifoaming agent is cyclomethicone.
